(19) United States
(12) Patent Application Publication
(10) Pub. No.: US 2014/0048072 A1
(43) Pub. Date: Feb. 20, 2014

(54) VENTILATOR-INITIATED PROMPT REGARDING DETECTION OF FLUCTUATIONS IN COMPLIANCE

(71) Applicant: Covidien LP, Boulder, CO (US)

(72) Inventors: Phyllis Angelico, Carlsbad, CA (US); Peter Doyle, Vista, CA (US)

(73) Assignee: Covidien LP, Boulder, CO (US)

(21) Appl. No.: 14/063,189

(22) Filed: Oct. 25, 2013

Related U.S. Application Data
(63) Continuation-in-part of application No. 12/955,523, filed on Nov. 29, 2010, now Pat. No. 8,595,639.

(51) Int. Cl. A61M 16/00

(52) U.S. Cl. CPC A61M 16/0051; A61M 16/0057
USPC 128/204.23; 128/204.21

(57) ABSTRACT

This disclosure describes systems and methods for monitoring and evaluating ventilatory data to provide useful notifications and/or recommendations. Indeed, many clinicians may not easily identify or recognize data patterns and correlations indicative of certain patient conditions or the effectiveness of ventilatory treatment. Further, clinicians may not readily determine appropriate adjustments that may address certain patient conditions or the effectiveness of ventilatory treatment. Specifically, clinicians may not readily detect or recognize the occurrence of fluctuations in compliance during various types of ventilation. According to embodiments, a ventilator may be configured to monitor and evaluate diverse ventilatory parameters to detect an occurrence of and potential causes for fluctuations in compliance and may subsequently issue suitable notifications and/or recommendations. The suitable notifications and/or recommendations may further be provided in a hierarchical format such that the clinician may selectively access information regarding the fluctuation in compliance and/or potential causes for the fluctuation in compliance.

### RELATED APPLICATIONS

This application is a continuation-in-part of U.S. patent application Ser. No. 12/955,523, entitled “Ventilator-Initiated Prompt Regarding Detection Of Fluctuations In Resistance,” filed Nov. 29, 2010, the specification of which is incorporated herein in its entirety.

### INTRODUCTION

A ventilator is a device that mechanically helps patients breathe by replacing some or all of the muscular effort required to inflate and deflate the lungs. In recent years, there has been an accelerated trend towards an integrated clinical environment. That is, medical devices are becoming increasingly integrated with communication, computing, and control technologies. As a result, modern ventilatory equipment has become increasingly complex, providing for detection and evaluation of a myriad of ventilatory parameters. However, due to the sheer magnitude of available ventilatory data, many clinicians may not readily identify certain patient conditions and/or changes in patient condition.

For example, during various types of volume or pressure ventilation, a fluctuation in compliance (e.g., an increase or a decrease) may be detected. A fluctuation in compliance may be indicative of a number of disparate patient and/or ventilator conditions, such as airway obstruction, the onset of asthma, acute respiratory distress syndrome (ARDS) or a pneumothorax. A clinician and/or ventilator system may be unable to determine the cause of a fluctuation in compliance. Thus, it may be difficult for a clinician and/or ventilator system to appropriately respond when potential causes for the fluctuation in compliance are unknown.

Indeed, clinicians and patients may greatly benefit from ventilator notifications when the ventilator detects certain patient conditions, changes in patient condition, effectiveness of ventilatory therapy, etc., based on an evaluation of available ventilatory data.

### Ventilator-Initiated Prompt Regarding Detection of Decreasing Compliance

This disclosure describes systems and methods for monitoring and evaluating ventilatory parameters, analyzing ventilatory data associated with those parameters, and providing useful notifications and/or recommendations to clinicians. Modern ventilators monitor, evaluate, and graphically represent a myriad of ventilatory parameters. However, many clinicians and/or ventilator systems may not easily identify or recognize data patterns and correlations indicative of certain patient conditions, changes in patient condition, and/or effectiveness of ventilatory treatment. Further, clinicians and/or ventilator systems may not readily determine appropriate ventilatory adjustments that may address certain patient conditions and/or the effectiveness of ventilatory treatment. Specifically, clinicians may not readily detect or recognize the cause of a detected fluctuation in compliance during various types of ventilation (e.g., volume control (VC) ventilation, pressure control (PC) ventilation, pressure support (PS) ventilation, volume-targeted-pressure-control (VC+), volume-targeted-pressure-support (VS) ventilation, proportional assist (PA) ventilation, etc.). According to embodiments, a ventilator may be configured to monitor and evaluate diverse ventilatory parameters to detect both the occurrence and potential causes for a fluctuation in compliance. Subsequently, the ventilator may issue suitable notifications and recommendations for addressing the fluctuation in compliance. The suitable notifications and recommendations may further be provided in a hierarchical format such that the clinician may selectively access information regarding the occurrence of a fluctuation in compliance, information regarding potential causes for the fluctuation in compliance, and/or information regarding one or more recommendations for addressing the fluctuation in compliance. In more automated systems, the one or more recommendations may be automatically implemented.

According to embodiments, a ventilator-implemented method for detecting a fluctuation in compliance during ventilation is provided. The method comprises receiving one or more ventilatory settings and collecting ventilatory data. The method further comprises processing the collected ventilatory data, wherein processing the collected ventilatory data includes determining a patient compliance. The method further comprises analyzing the compliance by comparing the patient compliance to a threshold compliance value and detecting a decrease in compliance upon determining that the patient compliance is less than the threshold compliance value. The method further includes detecting at least one secondary ventilatory parameter present when the patient compliance is determined. The method further includes displaying a smart prompt when a decrease in compliance is detected in conjunction with the at least one secondary ventilatory parameter.

According to additional embodiments, a ventilatory system for issuing a smart prompt when a decrease in compliance during ventilation is detected during ventilation is provided. The ventilatory system comprises at least one processor and at least one memory containing instructions that when executed by the at least one processor perform a method. The method comprises detecting a decrease in compliance during ventilation, detecting at least one secondary ventilatory parameter present when the patient compliance is detected, identifying one or more potential causes for an occurrence of a decrease in compliance in conjunction with at least one secondary ventilatory parameter, and determining one or more recommendations for addressing the decrease in compliance. The method further comprises displaying a smart prompt comprising one or more of: an alert regarding the decrease in compliance; a notification message displaying one or more secondary ventilatory parameters occurring concurrently with the decrease in compliance; and a recommendation message displaying the one or more recommendations for addressing the decrease in compliance.

According to additional embodiments, a graphical user interface for displaying one or more smart prompts corresponding to a detected condition is provided. The graphical user interface comprises at least one window and one or more elements within the at least one window. The one or more elements comprise at least one smart prompt element for communicating information regarding the detected condition, wherein the detected condition is a fluctuation in compliance in conjunction with one or more secondary ventilatory parameters.

According to additional embodiments, a ventilator processing interface for displaying a smart prompt in response to detecting a fluctuation in compliance in conjunction with one or more secondary ventilatory parameters is provided. The ventilator processing interface comprises means for retrieving at least some ventilatory data and means for detecting the decrease in compliance. The ventilator processing interface further comprises means for identifying one or more potential causes for the decrease in compliance based on one or more secondary ventilatory parameters present when the decrease in compliance is detected. The ventilator processing interface further comprises means for displaying the smart prompt comprising a notification message regarding the decrease in compliance and the one or more potential causes for the decrease in compliance.

According to embodiments, a ventilator-implemented method for detecting a fluctuation in compliance during ventilation is provided. The method comprises receiving one or more ventilatory settings and collecting ventilatory data. The method further comprises processing the collected ventilatory data, wherein processing the collected ventilatory data includes determining a patient compliance. The method further comprises analyzing the compliance by comparing the patient compliance to a threshold compliance value and detecting an increase in compliance upon determining that the patient compliance is more than the threshold compliance value. The method further includes detecting at least one secondary ventilatory parameter present when the patient compliance is determined. The method further includes displaying a smart prompt when an increase in compliance is detected in conjunction with the at least one secondary ventilatory parameter.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the claims.

### BRIEF DESCRIPTION OF THE DRAWINGS

The following drawing figures, which form a part of this application, are illustrative of described technology and are not meant to limit the scope of the claims in any manner, which scope shall be based on the claims appended hereto.

FIG. 1 is a diagram illustrating an embodiment of an exemplary ventilator connected to a human patient.

FIG. 2 is a block-diagram illustrating an embodiment of a ventilatory system for monitoring and evaluating ventilatory parameters to detect a fluctuation in compliance and to identify potential causes for the fluctuation in compliance.

FIG. 3 is a flow chart illustrating an embodiment of a method for detecting a fluctuation in compliance and issuing a suitable smart prompt.

FIG. 4 is a flow chart illustrating an embodiment of a method for detecting potential causes for a fluctuation in compliance and issuing a suitable smart prompt.

FIG. 5 is an illustration of an embodiment of a graphical user interface displaying a smart prompt element in a window having a notification regarding a decrease in compliance and regarding a potential cause for the decrease in compliance.

FIG. 6 is an illustration of an embodiment of a graphical user interface displaying an expanded smart prompt element in a window having a notification message regarding a decrease in compliance and a recommendation message regarding addressing the decrease in compliance.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment for alerting and advising clinicians and/or ventilatory systems regarding detected patient conditions.

This disclosure describes systems and methods for monitoring and evaluating ventilatory parameters, analyzing ventilatory data associated with those parameters, and providing useful notifications and/or recommendations to clinicians. Modern ventilators monitor, evaluate, and graphically represent a myriad of ventilatory parameters. However, many clinicians may not easily identify or recognize data patterns and correlations indicative of certain patient conditions, changes in patient condition, and/or effectiveness of ventilatory treatment. Further, clinicians may not readily determine appropriate ventilatory adjustments that may address certain patient conditions and/or the effectiveness of ventilatory treatment. Specifically, clinicians may not readily detect the causes of an occurrence of a fluctuation in compliance or identify potential causes for the fluctuation in compliance.

According to embodiments, a ventilator may be configured to monitor and evaluate diverse ventilatory parameters to detect an occurrence of a fluctuation in compliance and may identify potential causes for the fluctuation in compliance using at least one secondary ventilatory parameter. As used herein, a secondary ventilatory parameter may be defined as any other ventilatory and/or patient parameter detected, estimated, derived, or otherwise determined by the ventilatory system. Thereafter, the ventilator may issue suitable notifications regarding the occurrence of the fluctuation in compliance and may issue suitable recommendations based on the potential causes for the fluctuation in compliance. That is, the ventilator may detect a fluctuation in compliance based on, inter alia, ventilatory data (e.g., flow, volume, pressure, compliance, ventilator setup data, etc.), patient data (e.g., a patient body weight, a patient diagnosis, a patient gender, a patient age, etc.) and/or any suitable protocol, equation, etc. The ventilator may also detect one or more secondary ventilatory parameters occurring in conjunction with the fluctuation in compliance. Furthermore, the ventilator may determine one or more projected causes of the fluctuation in compliance (e.g., clogged expiratory filter, condensate accumulation in the ventilatory circuit, mucous plugging of the patient airway, over-distension of the lungs, Auto-PEEP, etc.) based on the detected secondary ventilatory parameter. Based on the one or more projected causes of the fluctuation in compliance, the ventilator may be configured to provide a notification and/or one or more recommendations for mitigating the fluctuation in compliance.

In some instances, the suitable notifications and recommendations may further be provided in a hierarchical format such that the clinician may selectively access information regarding the occurrence of a fluctuation in compliance, information regarding potential causes for the fluctuation in compliance, and/or information regarding one or more recommendations for addressing the fluctuation in compliance. In more automated systems, the one or more recommendations may be automatically implemented.

These and other embodiments will be discussed in further detail with reference to the following figures.

### Ventilator System

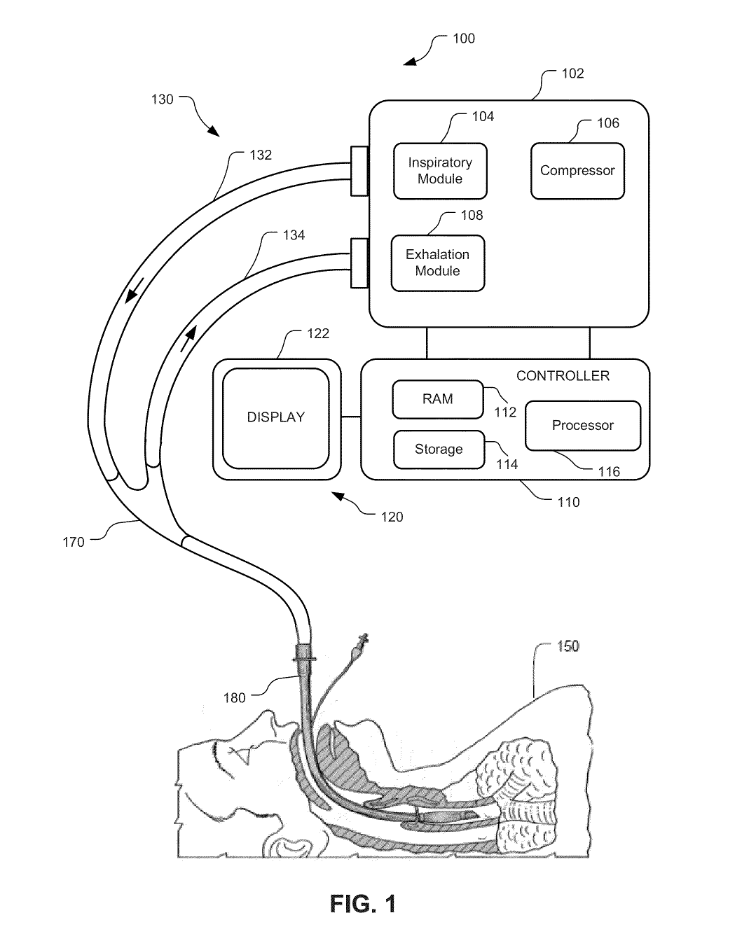

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, system 102 includes an exhalation module 108 coupled with the expiratory limb 134 and an inhalation module 104 coupled with the inspiratory limb 132. Compressor 106 or other source(s) of pressurized gases (e.g., air, oxygen, and/or helium) is coupled with inhalation module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.). Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

### Ventilator Components

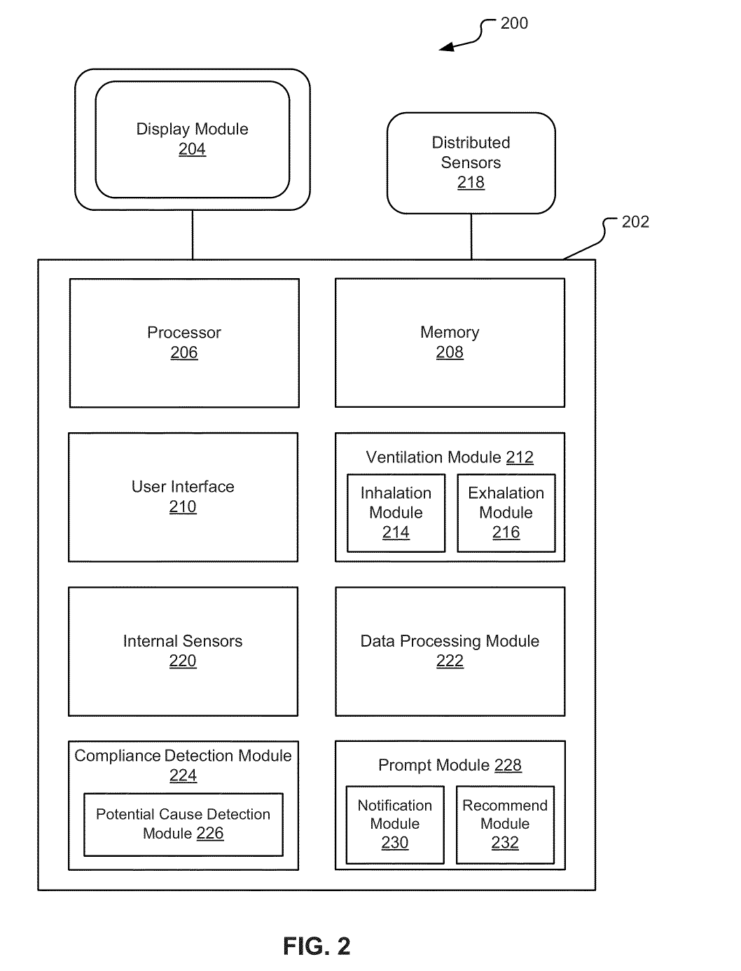

FIG. 2 is a block-diagram illustrating an embodiment of a ventilatory system for monitoring and evaluating ventilatory parameters to detect a fluctuation in compliance and to identify potential causes for the fluctuation in compliance.

Ventilatory system 200 includes ventilator 202 with its various modules and components. That is, ventilator 202 may further include, inter alia, memory 208, one or more processors 206, user interface 210, and ventilation module 212 (which may further include an inspiration module 214 and an exhalation module 216). Memory 208 is defined as described above for memory 112. Similarly, the one or more processors 206 are defined as described above for one or more processors 116. Processors 206 may further be configured with a clock whereby elapsed time may be monitored by the system 200.

The ventilatory system 200 may also include a display module 204 communicatively coupled to ventilator 202. Display module 204 provides various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, etc.). The elements may include controls, graphics, charts, tool bars, input fields, smart prompts, etc. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed to the clinician in the form of graphs, wave representations, pie graphs, or other suitable forms of graphic display. For example, one or more smart prompts may be displayed on the GUI and/or display module 204 upon detection of a fluctuation in compliance. Additionally or alternatively, one or more smart prompts may be communicated to a remote monitoring system coupled via any suitable means to the ventilatory system 200.

### Equation of Motion

Ventilation module 212 may oversee ventilation of a patient according to ventilatory settings. Ventilatory settings may include any appropriate input for configuring the ventilator to deliver breathable gases to a particular patient. Ventilatory settings may be entered by a clinician, e.g., based on a prescribed treatment protocol for the particular patient, or automatically generated by the ventilator, e.g., based on attributes (i.e., age, diagnosis, ideal body weight, gender, etc.) of the particular patient according to any appropriate standard protocol or otherwise. For example, ventilatory settings may include, inter alia, tidal volume (V_{T}), respiratory rate (RR), inspiratory time (T_{I}), inspiratory pressure (P_{I}), pressure support (P_{SUPP}), rise time percent (rise time %), peak flow, flow pattern, etc.

By way of general overview, the basic elements impacting ventilation may be described by the following ventilatory equation (also known as the Equation of Motion):

Pₘ+P_{ν}=V_{T}/C+R*F

During inspiration, P_{ν} represents the positive pressure delivered by a ventilator (generally in cm H₂O). Pₘ is a measure of muscular effort that is equivalent to the pressure generated by the muscles of a patient. If the patient's muscles are inactive, the Pₘ is equivalent to 0 cm H₂O. Alternatively, when the ventilator is not delivering positive pressure (i.e., P_{ν}=0 cm H₂O), Pₘ may be calculated according to the following formula:

Pₘ=V_{T}*E+R*F

As referenced in the above formulas, V_{T} represents the tidal volume delivered based on the pressure supplied, C refers to the compliance, E refers to elastance, R represents the resistance, and F represents the gas flow during inspiration (generally in liters per min (L/m)). According to some embodiments, Pₘ may be derived based on collected ventilatory data (see equation above). According to other embodiments, Pₘ may be measured directly by various distributed pressure sensors or otherwise. According to some embodiments, the ventilator may manipulate Pₘ data (either measured or derived) to estimate or quantify patient effort in terms of pressure (i.e., cmH₂O), in terms of a change in pressure over time (i.e., cmH₂O/s), or in terms of work (e.g., joules/liter (J/L)).

Alternatively, during exhalation, the Equation of Motion may be represented as:

Pₐ+Pₜ=V_{TE}/C+R*F

Here, Pₐ represents the positive pressure existing in the lungs (generally in cm H₂O), Pₜ represents the transairway pressure, V_{TE} represents the tidal volume exhaled, C refers to the compliance, R represents the resistance, and F represents the gas flow during exhalation (generally in liters per min (L/m)).

### Pressure

For positive pressure ventilation, pressure at the upper airway opening (e.g., in the patient's mouth) is positive relative to the pressure at the body's surface (i.e., relative to the ambient atmospheric pressure to which the patient's body surface is exposed, about 0 cm H₂O). As such, when P_{ν} is zero, i.e., no ventilatory pressure is being delivered, the upper airway opening pressure will be equal to the ambient pressure (i.e., about 0 cm H₂O). However, when inspiratory pressure is applied (i.e., positive pressure), a pressure gradient is created that allows gases to flow into the airway and ultimately into the lungs of a patient during inspiration (or, inhalation) until the pressure is equalized. When tidal volume (V_{T}) has been delivered to the lungs such that the inspiratory pressure is achieved and maintained, pressure is equalized and gases no longer flow into the lungs (i.e., zero flow).

Lung pressure or alveolar pressure, Pₐ, may be measured or derived. For example, Pₐ may be measured via a distal pressure transducer or other sensor near the lungs and/or the diaphragm. Alternatively, Pₐ may be estimated by measuring the plateau pressure, P_{Plat}, via a proximal pressure transducer or other sensor at or near the airway opening. Plateau pressure, P_{Plat}, refers to a slight plateau in pressure that is observed at the end of inspiration when inspiration is held for a period of time, sometimes referred to as an inspiratory hold or pause maneuver, or a breath-hold maneuver. That is, when inspiration is held, pressure inside the alveoli and mouth are equal (i.e., no gas flow). However, as a result of muscular relaxation and elastance of the lungs during the hold period, forces are exerted on the inflated lungs that create a positive pressure. This positive pressure is observed as a plateau in the pressure waveform that is slightly below the peak inspiratory pressure, P_{Peak}, prior to initiation of exhalation. As may be appreciated, for accurate measurement of P_{Plat}, the patient should be sedated or non-spontaneous (as muscular effort during the inspiratory pause may skew the pressure measurement). Upon determining P_{Plat} based on the pressure waveform or otherwise, P_{Plat} may be used as an estimate of Pₐ (alveolar pressure).

### Flow and Volume

Volume refers to the amount of gas delivered to a patient's lungs, usually in liters (L).

Flow refers to a rate of change in volume over time (F=ΔV/Δt). Flow is generally expressed in liters per minute (L/min or lpm) and, depending on whether gases are flowing into or out of the lungs, flow may be referred to as inspiratory flow (positive flow) or expiratory flow (negative flow), respectively. According to embodiments, the ventilator may control the rate of delivery of gases to the patient, i.e., inspiratory flow, and may control the rate of release of gases from the patient, i.e., expiratory flow.

As may be appreciated, volume and flow are closely related. That is, where flow is known or regulated, volume may be derived based on elapsed time. For example, during volume-controlled (VC) ventilation, a tidal volume, V_{T}, may be delivered upon reaching a set inspiratory time (T_{I}) at set inspiratory flow. Alternatively, set V_{T} and set inspiratory flow may determine the amount of time required for inspiration, i.e., T_{I}. During pressure control (PC) ventilation, pressure support (PS) ventilation, volume-targeted-pressure-control (VC+), volume-targeted-pressure-support (VS) ventilation, or proportional assist (PA) ventilation, delivered tidal volume may be determined based on integrating the flow waveform over T_{I} (set T_{I} in the case of PC or VC+ ventilation or patient-determined T_{I} in the case of PS, PA, and VS ventilation). For purposes of this disclosure, the terms “set V_{T}” or “target V_{T}” are used to refer to a ventilatory setting configured to deliver a particular volume of gases to a patient's lungs. Further, set V_{T} (or target V_{T}) may be configured by the clinician, automatically configured by the ventilator according to an appropriate protocol (e.g., based on one or more patient attributes including age, gender diagnosis, PBW or IBW), or otherwise.

### Compliance

Additional ventilatory parameters that may be measured and/or derived may include compliance and resistance, which refer to the load against which the patient and/or the ventilator must work to deliver gases to the lungs. Generally, compliance refers to a relative ease with which something distends and is the inverse of elastance, which refers to the tendency of something to return to its original form after being deformed. As related to ventilation, compliance refers to the lung volume achieved for a given amount of delivered pressure (C=ΔV/ΔP). Increased compliance may be detected when the ventilator measures an increased volume relative to the given amount of delivered pressure. Some lung diseases (e.g., acute lung injury (ALI), acute respiratory distress syndrome (ARDS), atelectasis, fibrosis, pulmonary edema, endotracheal tube displacement, pneumothorax, pneumonia, etc.) may decrease compliance and, thus, require increased pressure to inflate the lungs. Alternatively, other lung diseases may increase compliance, e.g., emphysema, and may require less pressure to inflate the lungs.

According to embodiments, static compliance and dynamic compliance may be calculated. Static compliance, C_{S}, represents compliance impacted by elastic recoil at zero flow (e.g., of the chest wall, patient circuit, and alveoli). As elastic recoil of the chest wall and patient circuit may remain relatively constant, static compliance may generally represent compliance as affected by elastic recoil of the alveoli. As described above, P_{Plat} refers to a slight plateau in pressure that is observed after relaxation of pleural muscles and elastic recoil, i.e., representing pressure delivered to overcome elastic forces. As such, P_{Plat} provides a basis for estimating C_{S} as follows:

C_{S}=V_{T}/(P_{Plat}−EEP)

Where V_{T} refers to tidal volume, P_{Plat} refers to plateau pressure, and EEP refers to end-expiratory pressure, or baseline pressure (including PEEP plus Auto-PEEP, if any), as discussed below. Note that proper calculation of C_{S} depends on accurate measurement of V_{T} and P_{Plat}.

Dynamic compliance, C_{D}, is measured during airflow and, as such, is impacted by both elastic recoil and airway resistance. Peak inspiratory pressure, P_{Peak}, which represents the highest pressure measured during inspiration, i.e., pressure delivered to overcome both elastic and resistive forces to inflate the lungs, is used to calculate C_{D} as follows:

C_{D}=V_{T}/(P_{Peak}−EEP)

Where V_{T} refers to tidal volume, P_{Peak} refers to peak inspiratory pressure, and EEP refers to end-expiratory pressure. According to embodiments, the term “compliance” may generally refer to dynamic compliance unless specified. According to embodiments, ventilatory data may be more readily available for trending compliance of non-triggering patients than of triggering patients.

### Resistance

Resistance refers to frictional forces that resist airflow, e.g., due to synthetic structures (e.g., endotracheal tube, exhalation valve, etc.), anatomical structures (e.g., bronchial tree, esophagus, etc.), or viscous tissues of the lungs and adjacent organs. Resistance is highly dependant on the diameter of the airway. That is, a larger airway diameter entails less resistance and a higher concomitant flow. Alternatively, a smaller airway diameter entails higher resistance and a lower concomitant flow. In fact, decreasing the diameter of the airway results in an exponential increase in resistance (e.g., two-times reduction of diameter increases resistance by sixteen times). As may be appreciated, resistance may also increase due to a restriction of the airway that is the result of, inter alia, increased secretions, bronchial edema, mucous plugs, bronchospasm, and/or kinking of the patient interface (e.g., invasive endotracheal or tracheostomy tubes).

Airway resistance may further be represented mathematically as:

R=Pₜ/F

Where Pₜ refers to the transairway pressure and F refers to the flow. That is, Pₜ refers to the pressure necessary to overcome resistive forces of the airway. Resistance may be expressed in centimeters of water per liter per second (i.e., cm H₂O/L/s).
Pulmonary Time Constant As discussed above, compliance refers to the lung volume achieved for a given amount of delivered pressure (C=ΔV/ΔP). That is, stated differently, volume delivered is equivalent to the compliance multiplied by the delivered pressure (ΔV=C*ΔP). However, as the lungs are not perfectly elastic, a period of time is needed to deliver the volume ΔV at pressure ΔP. A pulmonary time constant, τ, may represent a time necessary to inflate or exhale a given percentage of the volume at delivered pressure ΔP. The pulmonary time constant, τ, may be calculated by multiplying the resistance by the compliance (τ=R*C) for a given patient and τ is generally represented in seconds, s. The pulmonary time constant associated with exhalation of the given percentage of volume may be termed an expiratory time constant and the pulmonary time constant associated with inhalation of the given percentage of volume may be termed an inspiratory time constant.

According to some embodiments, when expiratory resistance data is available, the pulmonary time constant may be calculated by multiplying expiratory resistance by compliance. According to alternative embodiments, the pulmonary time constant may be calculated based on inspiratory resistance and compliance. According to further embodiments, the expiratory time, T_{E}, should be equal to or greater than a predetermined number of pulmonary time constants (e.g., about three pulmonary time constants) to ensure adequate exhalation. The predetermined number of pulmonary time constants may be selected via any suitable means, e.g., a standard protocol, an institutional protocol, clinician input, etc. According to embodiments, for a spontaneously-breathing patient, T_{E} (e.g., determined by trending T_{E} or otherwise) should be equal to or greater than the predetermined number of pulmonary time constants. For a non-spontaneously-breathing patient, set RR should yield a T_{E} that is equal to or greater than the predetermined number of pulmonary time constants.

### Normal Resistance and Compliance

According to embodiments, normal resistance and compliance may be determined based on a patient's predicted body weight (PBW) (or ideal body weight (IBW)). That is, according to a standardized protocol or otherwise, patient data may be compiled such that normal resistance and compliance values and/or ranges of values may be determined and provided to the ventilatory system. As such, a manufacturer, clinical facility, clinician, or otherwise, may configure the ventilator with normal resistance and compliance values and/or ranges of values based on PBWs (or IBWs) of a patient population. Thereafter, during ventilation of a particular patient, resistance and compliance data may be trended for the patient and compared to normal values and/or ranges of values based on the particular patient's PBW (or IBW). According to embodiments, the ventilator may give an indication to the clinician regarding whether the trended resistance and compliance data of the particular patient falls into normal ranges. According to some embodiments, data may be more readily available for trending resistance and compliance for non-triggering patients than for triggering patients.

According to further embodiments, a predicted T_{E} may be determined based on a patient's PBW (or IBW). That is, according to a standardized protocol or otherwise, patient population data may be compiled such that predicted T_{E} values and/or ranges of values may be determined based on PBWs (or IBWs) of the patient population and provided to the ventilatory system. Actual (or trended) T_{E} for a particular patient may then be compared to the predicted T_{E}. As noted previously, increased resistance and/or compliance may result in an actual T_{E} that is longer than predicted T_{E}. However, when actual T_{E} is consistent with predicted T_{E}, this may indicate that resistance and compliance for the particular patient fall into normal ranges.

According to further embodiments, a normal pulmonary time constant, τ, may be determined based on a patient's PBW (or IBW). That is, according to a standardized protocol or otherwise, patient data may be compiled such that normal τ values and/or ranges of values may be determined based on PBWs (or IBWs) of a patient population and provided to the ventilatory system. A calculated τ may be determined for a particular patient by multiplying resistance by compliance (as described above, resistance and compliance data may be more readily available for a non-triggering patient). As the product of resistance and compliance results in τ, increased resistance and/or compliance may result in an elevated τ value. However, when the calculated τ value for the particular patient is consistent with the normal τ value, this may indicate that the resistance and compliance of the particular patient fall into normal ranges.

### Patient Data

According to embodiments, patient data may be received by the ventilator 202. Patient data (including a patient diagnosis, a patient disability, a patient post-operative condition, a patient body weight, a patient gender, a patient age, etc.) may influence the ventilator's determination of the one or more causes for a fluctuation in compliance. Furthermore, patient data may influence the ventilator's determination of one or more appropriate recommendations for mitigating the fluctuation in compliance. As such, according to some embodiments, the ventilator may take into consideration patient data when determining potential causes and/or recommendations for a fluctuation in compliance.

Some patients may exhibit certain characteristics associated with various conditions and diseases, e.g., COPD, ARDS, post-operative condition (single lung, cardiac surgery), etc. For example, patients diagnosed with COPD may exhibit chronic elevated resistance due to constricted and/or collapsed airways, while ARDS patients may exhibit chronic elevated resistance due to an inflammatory condition of the airways. In some cases, patients diagnosed with various conditions and diseases associated with an obstructive component may exhibit elevated resistance over many months or years. According to some embodiments, patients having these conditions may also exhibit elevated compliance.

According to embodiments described herein, a clinician may input a patient diagnosis, e.g., COPD, ARDS, emphysema, etc. The ventilator may associate the patient diagnosis with certain lung and airway characteristics. For example, if the ventilator receives a patient diagnosis of COPD, the ventilator may associate this patient diagnosis with elevated resistance. The ventilator may further associate this patient diagnosis with an obstructive component. Alternatively, if the ventilator receives a patient diagnosis of emphysema, the ventilator may associate this patient diagnosis with elevated compliance. Alternatively still, a patient diagnosis of ARDS may be associated with increased resistance and/or decreased lung compliance.

### Inspiration

Ventilation module 212 may further include an inspiration module 214 configured to deliver gases to the patient according to prescribed ventilatory settings. Specifically, inspiration module 214 may correspond to the inhalation module 104 or may be otherwise coupled to source(s) of pressurized gases (e.g., air, oxygen, and/or helium), and may deliver gases to the patient. Inspiration module 214 may be configured to provide ventilation according to various ventilatory types and modes, e.g., via volume-targeted, pressure-targeted, or via any other suitable type of ventilation.

According to embodiments, the inspiration module 214 may provide ventilation via a form of volume ventilation. Volume ventilation refers to various forms of volume-targeted ventilation that regulate volume delivery to the patient. Different types of volume ventilation are available depending on the specific implementation of volume regulation. Volume ventilation may include volume-control (VC), volume-assist, or volume assist/control ventilation. Volume control (VC) ventilation may be provided by delivering a set peak flow and flow pattern for a period of time (T_{I}) to deliver a prescribed tidal volume (i.e., set V_{T}) to the patient. For non-spontaneously-breathing patients, a set V_{T} and inspiratory time (T_{I}) may be configured during ventilation start-up, e.g., based on the patient's predicted or ideal body weight (PBW or IBW). In this case, flow will be dependent on the set V_{T} and set T_{I}. Alternatively, set V_{T} and a peak flow and flow pattern may be set such that T_{I} is a function of these settings. For spontaneously-breathing patients, a set V_{T} may be configured and the patient may determine T_{I}.

According to embodiments, during volume ventilation, as volume and flow are regulated by the ventilator, delivered V_{T}, flow waveforms (or flow traces), and volume waveforms may be constant and may not be affected by variations in lung or airway characteristics (e.g., compliance and/or resistance). Alternatively, pressure readings may fluctuate based on lung or airway characteristics. According to some embodiments, the ventilator may control the inspiratory flow and then derive volume based on integrating the inspiratory flow over elapsed time.

According to alternative embodiments, the inspiration module 214 may provide ventilation via a form of pressure ventilation. Pressure-targeted types of ventilation may be provided by regulating the pressure delivered to the patient in various ways. According to embodiments described herein, pressure support (PS) ventilation and pressure control (PC) ventilation may be accomplished by setting an inspiratory pressure (P_{I}) (or a pressure support level, P_{SUPP}) for delivery to the patient. Pressure ventilation may also include volume-targeted-pressure-control (VC+) or volume-targeted-pressure-support (VS) ventilation, in which a set V_{T} is targeted by calculating and delivering an effective pressure at the patient airway. Furthermore, pressure ventilation may include proportional assist (PA) ventilation, in which a pressure is targeted that is a function of a clinician-selected percent support, PEEP, an estimate of the patient's resistance and elastance, and a calculation of tube resistance.

According to embodiments, during pressure control (PC) ventilation, the ventilator delivers mandatory breaths to a patient by “targeting” a pressure at the patient airway, which target pressure is equivalent to a set PEEP (if any) plus a set P_{I}. For example, the ventilator may increase pressure in the patient airway based on a set rise time %, which dictates how quickly the ventilator will generate the target pressure within a set T_{I}. The pressure trajectory for a PC breath type depends on the set P_{I}, set PEEP, set T_{I}, and the rise time %. In contrast, the flow-delivery profile is dependent on the rise time %, the patient's resistance and compliance, and the patient's inspiratory effort (if any). According to embodiments, during PC ventilation, the ventilator may further determine delivered V_{T} at the end of inspiration and compare the delivered V_{T} to a threshold V_{T} setting.

According to alternative embodiments, during volume-targeted-pressure-control (VC+) ventilation, the ventilator delivers mandatory breaths to a patient by calculating and delivering an effective pressure in the patient circuit that is projected to achieve a target tidal volume (V_{T}) within a set inspiratory time (T_{I}). More specifically, at the beginning of each breath, the ventilator may retrieve data regarding the end-inspiratory pressure (EIP), the end-expiratory pressure (EEP), and the delivered volume associated with the last breath cycle. For example, delivered volume (delivered V_{T}) may be determined based on integrating the net flow during the last inspiration and applying various volume compensations (e.g., tube compliance). Thereafter, the ventilator may utilize the retrieved data, the delivered V_{T}, and the patient's IBW or PBW to estimate the patient's compliance and may calculate a revised effective pressure for use in the next breathing cycle that is projected to deliver the set V_{T}. According to embodiments, during VC+ ventilation, the ventilator may further determine delivered V_{T} at the end of inspiration and compare the delivered V_{T} to a threshold V_{T} setting.

According to alternative embodiments, during pressure support (PS) ventilation, the ventilator delivers breaths spontaneously to a patient by “targeting” a pressure at the patient airway that is equivalent to a set PEEP plus a set pressure support (P_{SUPP}) level. For example, upon detection of an inspiratory effort the ventilator may increase pressure in the patient airway based on a set rise time % to achieve the target pressure. The pressure trajectory for a PS breath type depends on the set P_{SUPP}, set PEEP, and set rise time %. In contrast, the flow-delivery profile is a function of the rise time %, the patient's resistance and compliance, and the patient's inspiratory effort. According to embodiments, during PS ventilation, the ventilator may further determine delivered V_{T} at the end of inspiration and compare the delivered V_{T} to a threshold V_{T} setting.

According to alternative embodiments, during volume-targeted-pressure-support (VS) ventilation, the ventilator delivers spontaneous breaths to a patient by calculating and delivering an effective pressure in the patient circuit that is projected to achieve a set (or target) V_{T}. More specifically, at the beginning of each breath, the ventilator may retrieve data regarding the end-inspiratory pressure (EIP), the end-expiratory pressure (EEP), and the delivered volume associated with the last breath cycle. For example, delivered volume (delivered V_{T}) may be determined based on integrating the net flow during the last inspiration and applying various volume compensations (e.g., tube compliance). Thereafter, the ventilator may utilize the retrieved data, the delivered V_{T}, and the patient's IBW or PBW to estimate the patient's compliance and may calculate a revised effective pressure for use in the next breathing cycle that is projected to deliver the set V_{T}. According to embodiments, during VS ventilation, the ventilator may further determine delivered V_{T} at the end of inspiration and compare the delivered V_{T} to a threshold V_{T} setting.

According to still other embodiments, during proportional assist (PA) ventilation, the ventilator delivers a target pressure to the patient airway that is a function of a clinician-selected percent support, set PEEP, an estimate of the patient's resistance and elastance, and a calculation of the tube resistance (dependent on tube type and the internal diameter of the tube). According to embodiments, during PA ventilation, the ventilator may further determine delivered V_{T} at the end of inspiration and compare the delivered V_{T} to a threshold V_{T} setting.

According to further embodiments, the ventilator may be configured in various modes for delivering the various breath types. For example, in A/C mode, the ventilator may be configured to deliver VC, PC or VC+ breath types that are either initiated by the ventilator according to a set RR (e.g., ventilator-initiated-mandatory breaths or VIMs) or initiated by the patient based on detected inspiratory effort (e.g., patient-initiated-mandatory breaths or PIMs). According to an alternative example, in bi-level mode, the ventilator may alternate between high and low PEEP settings and may be configured to deliver PC, PA, or PS breath types, depending on whether the patient is spontaneously-breathing or not. Alternatively, in SIMV mode, the ventilator may be configured to deliver VC, PC or VC+ breath types during a mandatory interval (VIMs or PIMs) and to deliver either PA or PS breath types during a spontaneous interval. Alternatively still, in a spontaneous mode, the ventilator may be configured to deliver either PA or PS breath types to a spontaneously-breathing patient. Indeed, the ventilator may be configured to deliver pressure-based breaths according to any appropriate ventilatory mode or otherwise.

### Exhalation

Ventilation module 212 may further include an exhalation module 216 configured to release gases from the patient's lungs according to prescribed ventilatory settings. Specifically, exhalation module 216 may correspond to exhalation module 108 or may otherwise be associated with and/or control an exhalation valve for releasing gases from the patient. By way of general overview, a ventilator may initiate exhalation based on lapse of an inspiratory time setting (T_{I}) or other cycling criteria set by the clinician or derived from ventilator settings (e.g., detecting delivery of prescribed V_{T} or prescribed P_{I} based on a reference trajectory). Alternatively, exhalation may be cycled based on detection of patient effort or otherwise. Upon initiating the exhalation phase, exhalation module 216 may allow the patient to exhale by opening an exhalation valve. As such, exhalation is passive, and the direction of airflow, as described above, is governed by the pressure gradient between the patient's lungs (higher pressure) and the ambient surface pressure (lower pressure). Although expiratory flow is passive, it may be regulated by the ventilator based on the size of the exhalation valve opening. Indeed, the ventilator may regulate the exhalation valve in order to target set PEEP by applying a number of calculations and/or trajectories.

For a spontaneously-breathing patient, expiratory time (T_{E}) is the time from the end of inspiration until the patient triggers the next inspiration. For a non-spontaneously-breathing patient, it is the time from the end of inspiration until the next inspiration based on the set T_{I} and set RR. As may be further appreciated, at the point of transition between inspiration and exhalation, the direction of airflow may abruptly change from flowing into the lungs to flowing out of the lungs or vice versa depending on the transition. Stated another way, inspiratory flow may be measurable in the ventilatory circuit until P_{Peak} is reached (i.e., P_{I} plus PEEP or P_{SUPP} plus PEEP), at which point flow approximates zero. Thereafter, upon initiation of exhalation, expiratory flow is measurable in the ventilatory circuit until the pressure gradient between the lungs and the body's surface reaches zero (again, resulting in zero flow). However, in some cases, expiratory flow may still be positive, i.e., measurable, at the end of exhalation (termed positive end-expiratory flow or positive EEF). In this case, positive EEF is an indication that the pressure gradient has not reached zero or, similarly, that the patient has not completely exhaled.

### Ventilator Sensory Devices

The ventilatory system 200 may also include one or more distributed sensors 218 communicatively coupled to ventilator 202. Distributed sensors 218 may communicate with various components of ventilator 202, e.g., ventilation module 212, internal sensors 220, data processing module 222, compliance detection module 224, and any other suitable components and/or modules. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors 218 may further include pressure transducers that may detect changes in circuit pressure (e.g., electromechanical transducers including piezoelectric, variable capacitance, or strain gauge) or changes in a patient's muscular pressure (Pₘ). Distributed sensors 218 may further include various flowmeters for detecting airflow (e.g., differential pressure pneumotachometers). For example, some flowmeters may use obstructions to create a pressure decrease corresponding to the flow across the device (e.g., differential pressure pneumotachometers) and other flowmeters may use turbines such that flow may be determined based on the rate of turbine rotation (e.g., turbine flowmeters). Alternatively, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Indeed, any distributed sensory device useful for monitoring changes in measurable parameters during ventilatory treatment may be employed in accordance with embodiments described herein.

Ventilator 202 may further include one or more internal sensors 220. Similar to distributed sensors 218, internal sensors 220 may communicate with various components of ventilator 202, e.g., ventilation module 212, internal sensors 220, data processing module 222, compliance detection module 224, and any other suitable components and/or modules. Internal sensors 220 may employ any suitable sensory or derivative technique for monitoring one or more parameters associated with the ventilation of a patient. However, the one or more internal sensors 220 may be placed in any suitable internal location, such as, within the ventilatory circuitry or within components or modules of ventilator 202. For example, sensors may be coupled to the inhalation and/or exhalation modules for detecting changes in, circuit pressure and/or flow. Specifically, internal sensors may include pressure transducers and flowmeters for measuring changes in circuit pressure and airflow. Additionally or alternatively, internal sensors may utilize optical or ultrasound techniques for measuring changes in ventilatory parameters. For example, a patient's expired gases may be monitored by internal sensors to detect physiological changes indicative of the patient's condition and/or treatment, for example. Indeed, internal sensors may employ any suitable mechanism for monitoring parameters of interest in accordance with embodiments described herein.

As should be appreciated, with reference to the Equation of Motion, ventilatory parameters are highly interrelated and, according to embodiments, may be either directly or indirectly monitored. That is, parameters may be directly monitored by one or more sensors, as described above, or may be indirectly monitored by derivation according to the Equation of Motion.

### Ventilatory Data

Ventilator 202 may further include a data processing module 222. As noted above, distributed sensors 218 and internal sensors 220 may collect data regarding various ventilatory parameters. A ventilatory parameter refers to any factor, characteristic, or measurement associated with the ventilation of a patient, whether monitored by the ventilator or by any other device. Sensors may further transmit collected data to the data processing module 222 and, according to embodiments, the data processing module 222 may be configured to collect data regarding some ventilatory parameters, to derive data regarding other ventilatory parameters, and/or to graphically represent collected and derived data to the clinician and/or other modules of the ventilatory system. According to embodiments, any collected, derived, and/or graphically represented data may be defined as ventilatory data.

In some embodiments, some collected, derived, and/or graphically represented data may be indicative of end-expiratory flow (EEF), data regarding alveolar pressure Pₐ (e.g., via a breath-hold maneuver), P_{Peak} data, P_{Plat} data, volume data, flow trace data, EEP data, etc., may be collected, derived, and/or graphically represented by data processing module 220. Thereafter, the ventilatory data may be utilized by the ventilator to detect a fluctuation in compliance. Furthermore, the ventilatory data may be utilized by the ventilator to determine one or more potential causes for the fluctuation in compliance.

Some collected, derived, and/or graphically represented data may be indicative of a secondary ventilatory parameter. For instance, the collected, derived, and/or graphically represented data may be indicative of a delivered V_{T}. For example, delivered volume (delivered V_{T}) may be determined based on integrating the net flow during the last inspiration and applying various volume compensations (e.g., tube compliance). Furthermore, causes for fluctuations in compliance in conjunction with low delivered V_{T} may be determined. As such, ventilatory data that may be used to calculate the delivered V_{T}, to detect low-delivered V_{T} (e.g., based on a threshold V_{T} setting, protocol, or otherwise), and to identify potential causes for the fluctuation in compliance in conjunction with low-delivered V_{T} may be collected, derived, and/or graphically represented by data processing module 222.

Furthermore, according to embodiments, ventilatory data may also include ventilator setup data. For example, ventilator setup data may include data regarding whether the ventilator is configured to use a heated or non-heated humidifier or a heat and moisture exchanger (HME). Furthermore, ventilator setup data may include data regarding whether an inline nebulizer or closed suction catheter is being used for the patient. Indeed, ventilator setup data may include any data regarding the configuration of the ventilator, ventilator circuitry, patient interface, etc., that may be useful for characterizing a detected fluctuation in compliance to determine one or more potential causes for the fluctuation in compliance. For example, ventilator setup data may be useful in determining whether condensate is likely to accumulate in the patient circuit, etc.

Upon detecting a fluctuation in compliance, one or more secondary ventilatory parameters, and one or more potential causes for the fluctuation in conjunction with the detected secondary ventilatory parameter, the ventilator may determine one or more recommendations for mitigating the fluctuation in compliance based on, inter alia, ventilatory data, prescribed ventilatory settings, patient data, and/or any other suitable protocol, formula, equation, etc.

### Flow Data

According to embodiments, data processing module 222 may be configured to monitor inspiratory and expiratory flow. Flow may be measured by any appropriate, internal or distributed device or sensor within the ventilatory system. As described above, flowmeters may be employed by the ventilatory system to detect circuit flow. However, any suitable device either known or developed in the future may be used for detecting airflow in the ventilatory circuit.

Data processing module 222 may be further configured to plot monitored flow data graphically via any suitable means. For example, according to embodiments, flow data may be plotted versus time (flow waveform), versus volume (flow-volume loop), or versus any other suitable parameter as may be useful to a clinician. According to embodiments, flow may be plotted such that each breath may be independently identified. Further, flow may be plotted such that inspiratory flow and expiratory flow may be independently identified, e.g., inspiratory flow may be represented in one color and expiratory flow may be represented in another color.

As detailed above, compliance refers to the lung volume achieved for a given amount of delivered pressure. As may be appreciated, decreased compliance requires increased pressure to inflate the lungs. Generally, when a patient is intubated, i.e., having either an endotracheal or a tracheostomy tube in place, resistance is increased as a result of the smaller diameter of the tube over the patient's natural airway. Furthermore, compliance may be decreased when secretions, such as mucus, collect in the lungs or when a breathing tube has migrated to one side of the lungs (e.g., the right side). In addition, decreased compliance may be observed in patients with obstructive disorders, such as COPD, asthma, etc. When compliance decreases, additional tidal volume (V_{T}) may be delivered to the patient's lungs for a given amount of delivered pressure. According to embodiments, an evaluation of a flow trace and/or an evaluation of end-expiratory flow (EEF) may be used to detect a fluctuation in compliance, as described further herein.

### Pressure Data

According to embodiments, data processing module 222 may be configured to monitor pressure. Pressure may be measured by any appropriate, internal or distributed device or sensor within the ventilatory system. For example, pressure may be monitored by proximal electromechanical transducers connected near the airway opening (e.g., on the inspiratory limb, expiratory limb, at the patient interface, etc.). Alternatively, pressure may be monitored distally, at or near the lungs and/or diaphragm of the patient.

For example, P_{Peak} and/or P_{Plat} (estimating Pₐ) may be measured proximally (e.g., at or near the airway opening) via single-point pressure measurements. According to embodiments, Pₚₗₐₜ (estimating Pₐ) may be measured during an inspiratory pause maneuver (e.g., exhalation and inhalation valves are closed briefly at the end of inspiration for measuring the P_{Plat} at zero flow). According to other embodiments, circuit pressure may be measured during an expiratory pause maneuver (e.g., exhalation and inhalation valves are closed briefly at the end of exhalation for measuring EEP at zero flow). Alternatively, Pₘ may be distally measured (e.g., at or near the lungs and/or diaphragm) via multiple-point pressure measurements. Upon collecting Pₘ data, the ventilator may conduct calculations to quantify patient effort, which may be further used to estimate the patient's resistance and compliance. According to some embodiments, spontaneously-breathing patients may need to be sedated before taking some of the above-described pressure measurements.

Data processing module 222 may be further configured to plot monitored pressure data graphically via any suitable means. For example, according to embodiments, pressure data may be plotted versus time (pressure waveform), versus volume (pressure-volume loop or PV loop), or versus any other suitable parameter as may be useful to a clinician. According to embodiments, pressure may be plotted such that each breath may be independently identified. Further, pressure may be plotted such that inspiratory pressure and expiratory pressure may be independently identified, e.g., inspiratory pressure may be represented in one color and expiratory pressure may be represented in another color. According to additional embodiments, pressure waveforms and PV loops, for example, may be represented alongside additional graphical representations, e.g., representations of volume, flow, etc., such that a clinician may substantially simultaneously visualize a variety of parameters associated with each breath.

According to embodiments, PV loops may provide useful clinical and diagnostic information to clinicians regarding the resistance and/or compliance of a patient. Specifically, upon comparing PV loops from successive breaths, a change in compliance over time may be detected. For example, at constant pressure when compliance is decreasing, less volume is delivered to the lungs resulting in a shorter, wider PV loop. According to alternative embodiments, a PV loop may provide a visual representation indicative of compliance, that is, the area between the inspiratory plot of pressure vs. volume and the expiratory plot of pressure vs. volume. Thus, PV loops may also be compared to one another to determine whether compliance has changed over time. Additionally or alternatively, optimal compliance may be determined. That is, optimal compliance may correspond to the dynamic compliance determined from a PV loop during a recruitment maneuver, for example.

According to additional embodiments, PV curves may be used to compare C_{S} and C_{D} over a number of breaths. For example, a first PV curve may be plotted for C_{S} (based on P_{Plat} less EEP) and a second PV curve may be plotted for C_{D} (based on P_{Peak} less EEP). Under normal conditions, C_{S} and C_{D} curves may be very similar, with the C_{D} curve mimicking the C_{S} curve but shifted to the right (i.e., plotted at higher pressure). However, in some cases the C_{D} curve may flatten out and shift to the right relative to the C_{S} curve. This graphical representation may illustrate increasing Pₜ, and thus increasing R, which may be due to mucous plugging or bronchospasm, for example. In other cases, both the C_{D} curve and the C_{S} curves may flatten out and shift to the right. This graphical representation may illustrate an increase in P_{Peak} and P_{Plat}, without an increase in Pₜ, and thus may implicate a decrease in lung compliance, which may be due to tension pneumothorax, atelectasis, pulmonary edema, pneumonia, bronchial intubation, etc.

As may be further appreciated, relationships between resistance, static compliance, dynamic compliance, and various pressure readings may give indications of patient condition. For example, when C_{S} increases, C_{D} increases and, similarly, when R increases, C_{D} increases. Additionally, as discussed previously, Pₜ represents the difference in pressure attributable to resistive forces over elastic forces. Thus, where P_{Peak} and Pₜ are increasing with constant V_{T} delivery, R is increasing (i.e., where P_{Peak} is increasing without a concomitant increase in P_{Plat}). Where Pₜ is roughly constant, but where P_{Peak} and P_{Plat} are increasing with a constant V_{T} delivery, C_{S} is increasing.

### Volume Data

According to embodiments, data processing module 222 may be configured to derive volume via any suitable means. For example, as described above, during volume ventilation, a prescribed V_{T} may be set for delivery to the patient. In some cases, as a result of patient effort, the patient may “out-draw” the set V_{T}, resulting in a higher delivered V_{T} than the set V_{T}. Thus, for either volume or pressure ventilation, delivered V_{T} may be determined at the end of inspiration, i.e., by integrating net inspiratory flow over T_{I} (either set T_{I} or patient-determined T_{I}). Alternatively, expiratory flow may be monitored such that exhaled tidal volume (V_{TE}) may be derived by integrating net expiratory flow over expiratory time (T_{E}). In general, the delivered V_{T} should be completely exhaled and, thus, V_{TE} should be equivalent to delivered V_{T}. Indeed, delivered V_{T} may be determined via any suitable means, either currently known or developed in the future.

Data processing module 222 may be further configured to plot the volume data graphically via any suitable means. For example, according to embodiments, volume data may be plotted versus time (volume waveform), versus flow (flow-volume loop or FV loop), or versus any other suitable parameter as may be useful to a clinician. According to embodiments, volume may be plotted such that each breath may be independently identified. Further, volume may be plotted such that delivered V_{T} and V_{TE} may be independently identified, e.g., delivered V_{T} may be represented in one color and V_{TE} may be represented in another color. According to additional embodiments, volume waveforms and FV loops, for example, may be represented alongside additional graphical representations, e.g., representations of pressure, flow, etc., such that a clinician may substantially simultaneously visualize a variety of parameters associated with each breath.

### Compliance Fluctuation Detection

Ventilator 202 may further include a compliance detection module 224. As described above, compliance may be determined via any suitable means. Thereafter, detected compliance may be compared to a baseline compliance setting or range. Indeed, according to embodiments, the baseline compliance setting or range may be established according to any appropriate criteria (e.g., an appropriate standard, protocol, or otherwise) and may be configured by a manufacturer, an institution, a clinician, or otherwise. For instance, the baseline compliance setting may be prescribed by a physician or dictated by any suitable institutional or other protocol, based on, for example, a patient's physical characteristics. According to some embodiments, the clinician may not input the baseline compliance setting, but it may be automatically generated by the ventilator based on attributes of the patient (e.g., age, gender, diagnosis, PBW or IBW, etc.) or based on a default value. In some embodiments, based on PBW and/or other patient data, the compliance detection module 224 may identify a maximum compliance threshold and a minimum compliance threshold (e.g., based on clinician input, a standardized protocol, institutional protocol, etc.). For instance, in a ventilated patient, compliance may vary from 35 to 50 mL/cm H20 and may sometimes be higher (e.g., around 100 mL/cm H20). According to embodiments, the maximum and minimum compliance thresholds may define a range about a particular patient's measured and/or derived compliance. That is, for a patient exhibiting decreased compliance (e.g., a patient diagnosed with ARDS or ALI), the maximum and minimum compliance thresholds may be higher than for a patient exhibiting normal compliance.

To detect an occurrence of decreasing compliance, in some embodiments, the compliance detection module 224 may trend compliance values for the patient via any suitable means. “Trending,” as used herein, means collecting and/or deriving data over a plurality of breaths (or at predetermined intervals of time). For example, the compliance detection module 224 may calculate and trend compliance based on any suitable mathematical equation or formula (e.g., ΔV=C*ΔP). According to alternative embodiments, the compliance detection module 224 may evaluate PV loops based on one or more predetermined thresholds to detect whether compliance is decreasing, i.e., by comparing the area between the inspiratory plot of pressure versus volume and the expiratory plot of pressure versus volume over a number of breaths. According to alternative embodiments, compliance detection module 224 may evaluate PV curves to compare C_{S} and C_{D} over a number of breaths. That is, where both the C_{D} curve and the C_{S} curve straighten and shift to the left (e.g., illustrating increasing P_{Peak} and P_{Plat}) compliance may be decreasing. According to other embodiments, compliance may be determined and trended via any suitable means (via the compliance detection module 224 or any other ventilatory component).

The trended compliance may be compared to the baseline compliance value or range for the patient. Trended compliance data may be compared to, for example, a compliance threshold to detect a fluctuation in compliance. The compliance threshold may refer to a percentage increase or decrease in compliance (e.g., increase of 10%, 20%, 25%, 30%, or any other suitable percentage change). Alternatively, the compliance threshold may refer to a value increase or decrease in compliance (e.g., increase of 5 mL/cmH₂O, 10 mL/cmH₂O, or any other suitable value change). When the trended compliance data breaches a minimum or maximum threshold compliance value, the compliance detection module 224 may detect a fluctuation in compliance.

Compliance may fluctuate for a number of reasons, including changing lung conditions, improper body position, an onset of asthma, etc. Due to the variety of potential causes for a fluctuation in compliance, the compliance detection module 224 may further comprise a potential cause detection module 226. That is, the potential cause detection module 226 may evaluate various ventilatory data to determine potential causes for the fluctuation in compliance. In some embodiments, the potential cause determination module 226 may identify at least one secondary ventilatory parameter occurring when the fluctuation in compliance is detected. For example, the potential cause detection module 226 may determine whether the fluctuation in compliance occurred concurrently with an increase in resistance, an increase in patient inspiratory effort, etc. Other non-limiting examples of detected secondary ventilatory parameters may include a fluctuation in dynamic compliance C_{Dyn}, a fluctuation in tidal volume V_{T}, an increase in peak inspiratory pressure (PIP), an increase in mean airway pressure (MAP), a fluctuation in positive end expiratory pressure (PEEP), etc. Other detected secondary ventilatory parameters are contemplated.

As one example, the ventilator (e.g., via the potential cause determination module 226) may detect when the delivered V_{T} is less than a threshold V_{T}. According to alternative embodiments, low-delivered V_{T} may be detected when delivered V_{T} is less than the threshold V_{T} for a threshold time period (e.g., delivered V_{T} is greater than the threshold V_{T} for 2 consecutive breaths, for 3 of 5 consecutive breaths, for 30% of breaths over a period of time, etc.). Thus, if the fluctuation in compliance was detected concurrently with low-delivered V_{T} (e.g., during the previous 2 hours or since the start of ventilation, whichever is less), the potential cause detection module 226 may determine that the fluctuation in compliance was a potential cause for the low-delivered V_{T}.

According to embodiments, upon detecting at least one secondary ventilatory parameter, the ventilator may be configured with one or more potential causes associated with a fluctuation in compliance. For example, based on a protocol, standard, or otherwise, the ventilator may be configured to associate a fluctuation in compliance with one or more of the following potential causes, among others: worsening pneumonia, pneumothorax, hydrothorax, endotracheal tube migration, pleural effusion, acute lung injury (ALI), acute respiratory distress syndrome (ARDS), or inadvertent positive end-expiratory pressure (PEEP), patient body position, poor internal placement of the ventilatory circuit, etc. Indeed, the ventilator may be configured with any suitable number of potential causes associated with a fluctuation in compliance.

According to some embodiments, the ventilator may further evaluate patient data and/or a patient diagnosis to determine whether certain changed lung conditions are likely responsible for a fluctuation in compliance. For example, if patient data indicates that the patient is asthmatic, the ventilator may determine that bronchial constriction is a potential cause for a decrease in compliance. In contrast, if the patient has been diagnosed with ARDS, the ventilator may determine a higher relative likelihood that an increase in inflammation of the airways is a potential cause for the decrease in compliance.

According to some embodiments, the ventilator may display each of the one or more identified potential causes to the clinician. According to other embodiments, the ventilator may only display a subset of the one or more identified potential causes, e.g., only potential causes having higher relative likelihoods. The ventilator may be configured to determine and display the subset of identified potential causes via any suitable means. For example, the ventilator may be configured to display a predetermined number of the most likely potential causes, e.g., the three most likely potential causes. In this case, the ventilator will display the three most likely potential causes of three identified potential causes, of five identified potential causes, or of ten identified potential causes. As may be appreciated, any number of the most likely potential causes may be pre-configured, selected, or otherwise designated for display. According to alternative embodiments, the ventilator may be configured to display a most likely percentage of identified potential causes. For example, the ventilator may be configured to display the most likely 40% of potential causes. In this case, the most likely 4 of 10 identified potential causes, the most likely 2 of 5 identified potential causes, etc. (here, the ventilator may be further configured to round up or down to the nearest whole number of potential causes for display). According to still other embodiments, each potential cause may be designated with a likelihood probability upon evaluation of ventilatory data, patient data, statistical analyses, etc. (e.g., a likelihood probability between 1 and 100) and the ventilator may be configured to display only those potential causes with a likelihood probability greater than some number (e.g., a likelihood probability of 50 or more). Indeed, any suitable method for ranking, organizing, or otherwise identifying and displaying one or more likely potential causes for a decrease or an increase in compliance may be employed within the spirit of the present disclosure.

### Smart-Prompt Generation

Ventilator 202 may further include a smart prompt module 228. As described above, the occurrence of and potential causes for fluctuations in compliance may be very difficult for a clinician to detect. As may be appreciated, multiple ventilatory parameters may be monitored and evaluated in order to detect an occurrence of and potential causes for a fluctuation in compliance. As such, upon detection of a fluctuation in compliance, the smart prompt module 228 may be configured to notify the clinician that a fluctuation in compliance has occurred and/or to provide one or more potential causes for the fluctuation in compliance. Furthermore, the ventilator may provide one or more suggestions or recommendations for addressing the fluctuation in compliance. For example, smart prompt module 228 may be configured to notify the clinician by displaying a smart prompt on display monitor 204 and/or within a window of the GUI. According to additional embodiments, the smart prompt may be communicated to and/or displayed on a remote monitoring system communicatively coupled to ventilatory system 200. Alternatively, in an automated embodiment, the smart prompt module 228 may communicate with a ventilator control system so that the one or more recommendations may be automatically implemented to address the fluctuation in compliance.

In order to accomplish the various aspects of the notification and/or recommendation message display, the smart prompt module 228 may communicate with various other components and/or modules. For instance, smart prompt module 228 may be in communication with data processing module 222, compliance detection module 224, potential cause detection module 226, or any other suitable module or component of the ventilatory system 200. That is, smart prompt module 228 may receive an indication that a fluctuation in compliance has been detected by any suitable means. In addition, smart prompt module 228 may receive information regarding one or more potential causes for the fluctuation in compliance. Further still, smart prompt module 228 may determine and offer one or more recommendations for addressing the fluctuation in compliance.

Smart prompt module 228 may further comprise additional modules for making notifications and/or recommendations to a clinician regarding the occurrence of a fluctuation in compliance. For example, according to embodiments, smart prompt module 228 may include a notification module 230 and a recommendation module 232. For instance, smart prompts may be provided according to a hierarchical structure such that a notification message and/or a recommendation message may be initially presented in summarized form and, upon clinician selection, an additional detailed notification and/or recommendation message may be displayed. According to alternative embodiments, a notification message may be initially presented and, upon clinician selection, a recommendation message may be displayed. Alternatively or additionally, the notification message may be simultaneously displayed with the recommendation message in any suitable format or configuration.

Specifically, according to embodiments, the notification message may alert the clinician as to the detection of a patient condition, a change in patient condition, or an effectiveness of ventilatory treatment. For example, the notification message may alert the clinician that a decrease in compliance has been detected. The notification message may further alert the clinician regarding potential causes for the decrease in compliance (e.g., low-delivered V_{T} detected concurrent with a decrease in dynamic lung/chest wall compliance, increasing peak inspiratory pressure detected concurrent with a decrease in dynamic lung/chest wall compliance, etc.). The notification message may further be specific to a particular type of ventilation. For instance, if the ventilator is delivering PC or PS ventilation, the ventilator may provide a secondary notification message upon detecting a decrease in compliance: “Decreasing V_{T} Detected.” If the ventilator is delivering VC or VC+ ventilation, the ventilator may provide a secondary notification message upon detecting a decrease in compliance: “Increasing PIP Detected.”

Additionally, according to embodiments, the recommendation message may provide various suggestions to the clinician or ventilatory system for addressing a detected condition.

For instance, upon detecting a fluctuation in compliance in conjunction with at least one secondary ventilatory parameter, the ventilator may provide the recommendation: “Consider: (1) checking patient for barotrauma; (2) fluid in the lungs.” The ventilator may provide recommendations: “Consider: (1) checking for pneumonia or worsening pneumonia; (2) checking for endotracheal tube migration; (3) checking for pleural effusion; (4) checking for potential ALI; (5) checking for potential ARDS.” Such recommendations may be provided if the ventilator is delivering any type of ventilation (e.g., PC, PS, VC, VC+, VS ventilation, etc.).

As described above, smart prompt module 228 may also be configured with notification module 230 and recommendation module 232. The notification module 230 may be in communication with data processing module 222, the compliance detection module 224, potential cause detection module 226, or any other suitable module or component to receive an indication that a fluctuation in compliance has been detected and identification of one or more potential causes for the fluctuation in compliance. Notification module 230 may be responsible for generating a notification message via any suitable means. For example, the notification message may be provided as a tab, banner, dialog box, or other similar type of display. Further, the notification message may be provided along a border of the graphical user interface, near an alarm display or bar, or in any other suitable location. A shape and size of the notification message may further be optimized for easy viewing with minimal interference to other ventilatory displays. The notification message may be further configured with a combination of icons and text such that the clinician may readily identify the message as a notification message. The notification message may further be associated with a primary prompt.

The recommendation module 232 may be responsible for generating one or more recommendation messages via any suitable means. The one or more recommendation messages may provide suggestions and information regarding addressing a detected condition and may be accessible from the notification message. For example, the one or more recommendation messages may provide suggestions for adjusting one or more ventilatory parameters to address the detected condition, may provide suggestions for checking ventilatory equipment or the patient, or may provide other helpful information. Specifically, the one or more recommendation messages may provide suggestions and information regarding addressing decreasing compliance. The one or more recommendation messages may further be associated with a secondary prompt.

As noted above, according to embodiments, the notification message may be associated with a primary prompt and the one or more recommendation messages may be associated with a secondary prompt. That is, a primary prompt may provide an alert that decreasing compliance has been detected and may further provide one or more potential causes for the decreasing compliance. Alternatively, an alert may be separately provided, indicating that decreasing compliance was detected, and the primary prompt may provide the one or more potential causes for the decreasing compliance. According to additional or alternative embodiments, the secondary prompt may provide the one or more recommendations and/or information that may aid the clinician in further addressing and/or mitigating the detected condition. For example, the secondary prompt may recommend addressing the decreasing compliance by checking the patient for one or more potential conditions (e.g., barotrauma, fluid in the lungs, etc.), by investigating causes for low-delivered V_{T} or increasing PIP, etc. According to further embodiments, a single smart prompt may be displayed (i.e., not configured with a primary prompt and a secondary prompt) and may include at least one of: a notification that a decrease or an increase in compliance has occurred, one or more potential causes for the decrease or increase in compliance, and/or one or more recommendations for addressing the decrease or increase in compliance. According to alternative embodiments, the secondary prompt described above may be provided as the primary prompt and the primary prompt described above may be provided as the secondary prompt.

Smart prompt module 228 may also be configured such that smart prompts (including alerts, primary prompts, and/or secondary prompts) may be displayed in a partially transparent window or format. The transparency may allow for notification and/or recommendation messages to be displayed such that normal ventilator GUI and ventilatory data may be visualized behind the messages. As described previously, notification and/or recommendation messages may be displayed in areas of the display screen that are either blank or that cause minimal distraction from the ventilatory data and other graphical representations provided by the GUI. However, upon selective expansion of a smart prompt, ventilatory data and graphs may be at least partially obscured. As a result, translucent display may provide the smart prompt such that it is partially transparent. Thus, graphical and other data may be visible behind the smart prompt.

Additionally, alerts, primary prompts, and/or secondary prompts may provide immediate access to the display and/or settings screens associated with the detected condition. For example, an associated parameter settings screen may be accessed from a smart prompt via a hyperlink such that the clinician may address the detected condition as necessary. An associated parameter display screen may also be accessed such that the clinician may view clinical data associated with the detected condition in the form of charts, graphs, or otherwise. For example, when a decrease in compliance has been detected, depending on the one or more potential causes for the decrease in compliance, the clinician may be able to access ventilatory settings or perform a patient examination to address the potential causes for a decrease in compliance and/or to view ventilatory data associated with the one or more potential causes for a decrease in compliance (e.g., charts displaying historical data and/or graphics displaying historical flow waveforms, volume waveforms, and/or pressure waveforms that implicated decreased compliance.

According to embodiments, upon viewing a smart prompt (including any associated alert, primary prompt, and/or secondary prompt), upon addressing the detected condition by adjusting one or more ventilatory settings or otherwise, or upon manual selection, the smart prompt may be cleared from the GUI. For example, according to some embodiments, upon receiving a ventilatory settings change, the ventilator may reset detection of a fluctuation in compliance when two consecutive breaths exhibit compliance than the compliance baseline value or threshold setting or when all breaths over the previous 30 seconds exhibit compliance less than the compliance baseline value or threshold setting. According to alternative embodiments, in the absence of user activity, the ventilator may reset detection of a fluctuation in compliance when all breaths over the previous 60 seconds exhibit compliance less than the compliance baseline value or threshold setting. Thereafter, upon resetting detection of a fluctuation in compliance, the ventilator may clear the smart prompt from the GUI and resume evaluation of ventilatory data by the compliance detection module 224 and the potential cause detection module 226.

### Compliance Detection and Notification

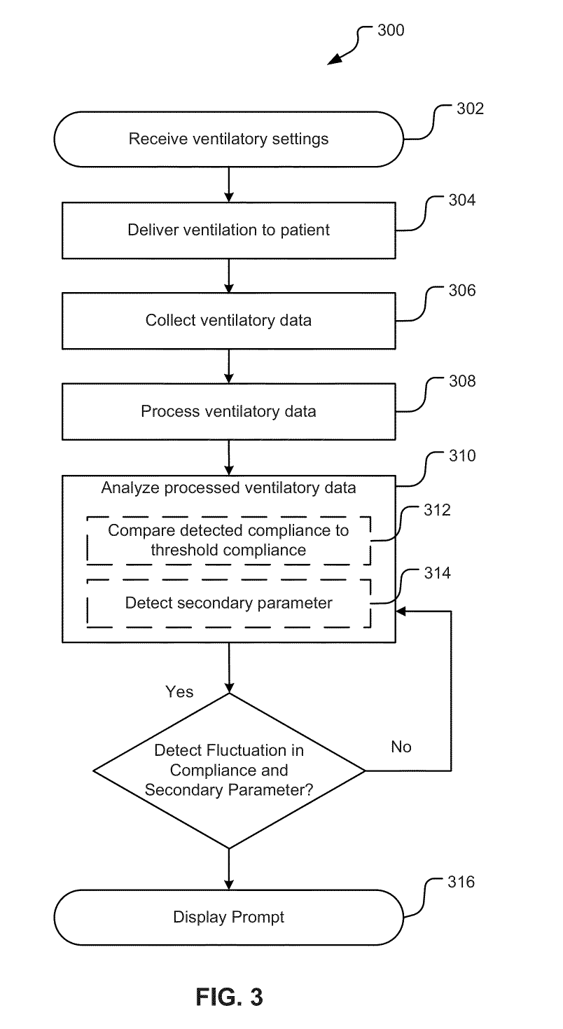

FIG. 3 is a flow chart illustrating an embodiment of a method for detecting a fluctuation (e.g., an increase or a decrease) in compliance and issuing a suitable smart prompt.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

The illustrated embodiment of the method 300 depicts a method for detecting a fluctuation in compliance that may be associated with one or more patient conditions. According to embodiments described herein, ventilation delivered may generally include volume control (VC) ventilation, pressure control (PC) ventilation, pressure support (PS) ventilation, volume-targeted-pressure-control (VC+), volume-targeted-pressure-support (VS), proportional assist (PA) ventilation, etc.

Method 300 begins with a receive settings operation 302. For example, at receive settings operation 302, the ventilator may receive one or more ventilatory settings associated with a type of ventilation (e.g., VC, PC, PS, VC+, VS, or PA ventilation). For example, according to embodiments, the ventilator may be configured to provide VC ventilation to a patient. As such, the ventilatory settings may include a respiratory rate (RR), an inspiratory time (T_{I}), a patient PBW or IBW, PEEP, PIP, a threshold V_{T}, a threshold compliance, etc. According to alternative embodiments, the ventilator may be configured to provide PC ventilation to a patient. As such, the ventilatory settings may include an inspiratory pressure (P_{I}), a respiratory rate (RR), an inspiratory time (T_{I}), a patient PBW or IBW, PEEP, a threshold V_{T}, a threshold compliance, rise time %, etc. According to alternative embodiments, the ventilator may be configured to provide PS ventilation to a patient. As such, the ventilatory settings and/or input received may include a pressure support setting (P_{SUPP}), a patient PBW or IBW, PEEP, a threshold V_{T}, a threshold compliance, a rise time %, etc. According to alternative embodiments, the ventilator may be configured to provide VC+ ventilation to a patient. As such, the ventilatory settings may include an inspiratory pressure (P_{I}), a respiratory rate (RR), an inspiratory time (T_{I}), a set (or target) V_{T}, a patient PBW or IBW, PEEP, PIP, a threshold V_{T}, a threshold compliance, rise time %, etc. According to alternative embodiments, the ventilator may be configured to provide VS ventilation to a patient. As such, the ventilatory settings may include a pressure support setting (P_{SUPP}), a set (or target) V_{T}, a patient PBW or IBW, PIP, a threshold V_{T}, a threshold compliance, rise time %, etc. According to still alternative embodiments, the ventilator may be configured to provide PA ventilation to a patient. As such, the ventilatory settings may include a percent support setting, a patient PBW or IBW, PEEP, a threshold V_{T}, a threshold compliance, tube type and internal diameter (I.D.), etc.

According to some embodiments, the clinician may select one or more of the ventilatory settings from a range of options. Alternatively, one or more of the ventilatory settings may be automatically generated by the ventilator based on a default value or based on one or more attributes of the patient (e.g., age, gender, diagnosis, PBW or IBW, etc.). For example, according to some embodiments, the threshold compliance setting may be selectable by a clinician between 30 to 50 mL/cm H20, with an automatic default value of 30 mL/cm H20. According to alternative embodiments, the selectable range for the threshold compliance setting may be any suitable range (e.g., between 35 to 50 mL/cm H20) and the default value may be any suitable value (e.g., 30 mL/cm H20, 35 mL/cm H20, 40 mL/cm H20, etc.). Alternatively still, the threshold compliance setting may be automatically generated by the ventilator based on one or more patient attributes or otherwise.

At deliver ventilation operation 304, the ventilator provides ventilation to the patient, as described above. That is, according to embodiments, the ventilator may deliver VC, PC, PS, VC+, VS, or PA breath types to a patient. According to additional embodiments, the ventilator may deliver breath types to the patient according to various ventilatory modes (e.g., A/C, spontaneous, BiLevel, SIMV, etc.). For example, during VC ventilation, the ventilator may deliver a set peak flow and flow pattern for a period of time, i.e., set inspiratory time (T_{I}). Based on the set peak flow, flow pattern and patient inspiratory effort (if any), a volume of gases will be delivered to the patient's lungs (i.e., delivered V_{T}). For example, during PC or VC+ ventilation, the ventilator may deliver an effective pressure (equivalent to PEEP plus set P_{I}) at the patient airway for a period of time, i.e., set inspiratory time (T_{I}). Based on the effective pressure, resistance, compliance and patient inspiratory effort (if any), a volume of gases will be delivered to the patient's lungs (i.e., delivered V_{T}). Alternatively, during PS or VS ventilation, the ventilator may deliver an effective pressure (equivalent to PEEP plus set P_{SUPP}) at the patient airway. Based on the effective pressure, resistance, compliance and patient inspiratory effort, a volume of gases will be delivered to the patient's lungs (i.e., delivered V_{T}). Alternatively still, during PA ventilation, the ventilator may target a pressure at the patient airway that is a function of the percent support, PEEP, an estimate of the patient's resistance and elastance, and a calculation of the tube resistance. Based on the target pressure, resistance, compliance, and patient inspiratory effort, a volume of gases will be delivered to the patient's lungs (i.e., delivered V_{T}). Furthermore, the ventilator may initiate an exhalation phase when a set T_{I} has been reached, when patient exhalation cycling is detected, or based on any other appropriate cycling criterion.

At collect ventilatory data operation 306, the ventilator may collect various ventilatory data associated with ventilation of a patient. For example, as described above, the ventilator may collect ventilatory data regarding flow and pressure parameters. The ventilator may collect the ventilatory data via any suitable means, e.g., any internal or distributed sensor including flowmeters, pressure transducers, etc. Ventilatory data may be collected from sources external to the ventilator (e.g., esophageal balloon, EIT, etc.).

At process ventilatory data operation 308, the ventilator may conduct various data processing operations. For example, at data processing operation 308, the ventilator may derive various ventilatory data associated with the ventilation of a patient. For example, as described above, the ventilator may collect ventilatory data regarding flow and pressure parameters. Additionally, the ventilator may calculate or derive ventilatory data based on the collected data, e.g., delivered volume, resistance, compliance, patient effort, etc. For example, compliance may be calculated or otherwise derived from collected data such as flow, pressure, or volume (e.g., C=ΔV/ΔP). Furthermore, compliance may be derived (e.g., trended) over a plurality of breaths or at predetermined intervals of time. Delivered volume (delivered V_{T}) may be determined based on integrating the net flow during the last inspiration and applying various volume compensations (e.g., tube compliance). Additionally, the ventilator may generate various graphical representations of the collected and/or derived ventilatory data, e.g., including charts, graphs depicting flow waveforms, pressure waveforms, pressure-volume loops, flow-volume loops, or other suitable data representations.

At analyze operation 310, the ventilator may evaluate the processed ventilatory data to determine whether a certain patient condition exists. For example, according to embodiments, the ventilator may analyze the compliance value and the secondary ventilatory parameter (e.g., delivered V_{T}) in light of a threshold compliance value and a secondary parameter setting (e.g., a threshold V_{T}). As described above, the threshold compliance value and a threshold secondary parameter value may be received as input from the clinician or may be automatically generated by the ventilator based on a default value or based on the patient's PBW or other appropriate criteria (e.g., based on a suitable protocol or otherwise). According to embodiments, the ventilator may analyze the ventilatory data by comparing the detected compliance to the threshold compliance via any suitable means.

Analyze operation 310 may further include compare compliance operation 312 and detect secondary parameter operation 314. At compare compliance operation 312, the ventilator may determine whether a fluctuation in compliance has occurred. For example, upon comparing the derived compliance to the threshold compliance in the analyze operation above, the ventilator may determine that derived compliance is greater or less than than the threshold compliance and the ventilator may detect a fluctuation in compliance. Alternatively, according to some embodiments, the ventilator may determine that a fluctuation in compliance occurred when a derived compliance is more or less than the threshold compliance over a threshold time period (e.g., a derived compliance is more or less than the threshold compliance for 2 consecutive breaths, for 3 of 5 consecutive breaths, for 30% of breaths over a period of time (e.g., hours), etc.). If a fluctuation in compliance is detected, the operation may proceed to compare secondary parameter operation 314. If a fluctuation in compliance is not detected, the operation may return to analyze operation 310.

At detect secondary parameter operation 314, the ventilator may determine whether a second parameter is occurring during a fluctuation in compliance. For example, upon comparing the derived or detected compliance to a threshold compliance in the analyze operation above, the ventilator may also determine if a delivered V_{T} is less than a threshold V_{T}. If the delivered V_{T} is less than a threshold V_{T}, the ventilator may determine that low-delivered V_{T} is occurring in conjunction with a decrease in compliance. Comparison of delivered V_{T} to the threshold V_{T} may be performed over a threshold time period (e.g., delivered V_{T} is less than the threshold V_{T} for 2 consecutive breaths, for 3 of 5 consecutive breaths, for 30% of breaths over a period of time, etc.). Alternatively or additionally, according to some embodiments, the ventilator may determine that increased PIP has occurred, increased MAP has occurred, or decreased C_{d} has occurred. If a secondary parameter is detected, the operation may proceed to display smart prompt operation 316. If the secondary parameter is not detected, the operation may return to analyze operation 310, or may display a smart prompt indicating the occurrence of a fluctuation in compliance.

At display smart prompt operation 316, the ventilator may alert the clinician via any suitable means that a fluctuation in compliance in conjunction with the presence of a secondary parameter was detected. For example, according to embodiments, the ventilator may display a smart prompt including a notification message and/or one or more recommendation messages regarding the detection of a decrease in compliance in conjunction with low-delivered V_{T} on the GUI. According to alternative embodiments, the ventilator may communicate the smart prompt, including the notification message and/or the one or more recommendation messages, to a remote monitoring system communicatively coupled to the ventilator. According to some embodiments, the fluctuation in compliance may fall within acceptable predetermined ranges such that the ventilator does not issue an alarm upon detecting the fluctuation in compliance. That is, the fluctuation in compliance may be detected for purposes of generating a smart prompt, but may not rise to the level of alarm generation.

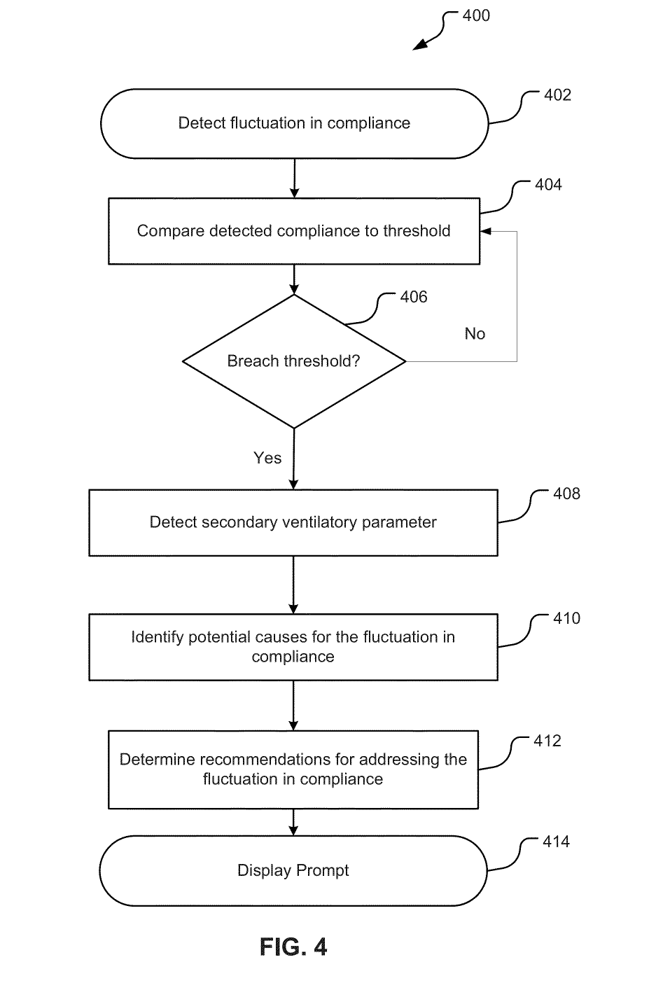

FIG. 4 is a flow chart illustrating an embodiment of a method for detecting potential causes for a fluctuation in compliance and issuing a suitable smart prompt.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

The illustrated embodiment of the method 400 depicts a method for issuing a smart prompt upon detecting a fluctuation in compliance. Method 400 begins with detect operation 402, wherein the ventilator detects that a fluctuation in compliance has occurred, as described above in method 300.

At compare operation 404, the ventilator may compare detected compliance to a threshold. Compliance data may be compared to, for example, a compliance threshold to detect a fluctuation in compliance. The compliance threshold may refer to a percentage change in compliance (e.g., an increase or decrease of 10%, 20%, 25%, 30%, or any other suitable percentage). Alternatively, the compliance threshold may refer to a value change in compliance (e.g., an increase or decrease of 5 mL/cmH₂O, 10 mL/cmH₂O, or any other suitable value). Furthermore, the compliance threshold may involve a time component (e.g., increase or decrease over a 2 hour period, from start of ventilation, or over a particular number of breaths). Indeed, according to embodiments, the compliance threshold may be established according to any appropriate criteria (e.g., an appropriate standard, protocol, or otherwise) and may be configured by a manufacturer, an institution, a clinician, or otherwise.

At breach threshold determination operation 406, the ventilator may determine whether the compared ventilatory data breaches one or more thresholds. For example, when the compliance data breaches the compliance threshold, the ventilator may detect a fluctuation in compliance. If the ventilator determined that the compared ventilatory data breached one or more thresholds, the operation may proceed to derive secondary ventilatory parameters operation 408. If the ventilator determined that the compared ventilatory data did not breach one or more thresholds, the operation may return to retrieve ventilatory data operation 404.

At derive secondary ventilatory parameters operation 408, the ventilator may derive various secondary ventilatory parameters such as delivered V_{T}, PIP, or MAP. Secondary parameters may be derived from collected flow, pressure, and/or volume data. Secondary parameters may be collected from sources external to the ventilator (e.g., esophageal balloon, EIT, etc.). However, any other ventilatory parameters may be retrieved that may be indicative of other potential causes for a fluctuation in compliance.

At identify operation 410, the ventilator may determine one or more potential causes for the fluctuation in compliance. For example, if the ventilator detected a decrease in compliance concurrently with the low-delivered V_{T}, the ventilator may determine that the decrease in compliance and the low-delivered V_{T} are an indication that one or more conditions may be present in the patient. For instance, if the compliance data breached the compliance threshold over the previous 2 hours or since the start of ventilation (whichever is less), and the ventilator determines that the decrease in compliance was detected concurrently with the low-delivered V_{T}, the ventilator may identify a potential cause for the occurrence of both simultaneously (or substantially simultaneously). Alternatively, if the ventilator detected an increase in PIP concurrently with the detected decrease in compliance, the ventilator may determine one or more potential causes for the occurrence of both simultaneously (or substantially simultaneously).

At determine operation 412, the ventilator may determine one or more recommendations for addressing the fluctuation in compliance. For instance, the recommendation message may provide various suggestions to the clinician or ventilatory system for addressing the detected fluctuation in compliance in conjunction with at least secondary ventilatory parameter. For instance, upon detecting a decrease in compliance in conjunction with at least one secondary ventilatory parameter, the ventilator may provide the recommendation: “Consider: (1) checking patient for barotrauma; or (2) checking for fluid in the lungs.” The ventilator may provide recommendations: “Consider: (1) checking for pneumonia or worsening pneumonia; (2) checking for endotracheal tube migration; (3) checking for pleural effusion; (4) checking for potential ALI; (5) checking for potential ARDS.” Such recommendations may be provided if the ventilator is delivering any type of ventilation (e.g., PC, PS, VC, VC+, VS ventilation, etc.).

At display smart prompt operation 414, the ventilator may alert the clinician via any suitable means that a fluctuation in compliance was detected. For example, according to embodiments, a smart prompt may include an appropriate primary prompt and an appropriate secondary prompt. Additionally or alternatively, the appropriate primary prompt may include an appropriate notification message that a fluctuation in compliance was detected and may include the one or more potential causes for the fluctuation in compliance. According to alternative embodiments, the notification message may be separately displayed from the one or more potential causes for the fluctuation in compliance. According to this embodiment, the notification message may be initially displayed and the one or more potential causes may be optionally displayed upon selection or activation by the clinician. According to further embodiments, the appropriate secondary prompt may provide the one or more recommendations for addressing the fluctuation in compliance. According to some embodiments, the appropriate primary prompt may be initially displayed and the appropriate secondary prompt may be optionally displayed upon selection or activation by a clinician. The smart prompt (including the appropriate primary prompt and/or the appropriate secondary prompt) may be displayed via any suitable means, e.g., on the ventilator GUI and/or at a remote monitoring station, such that the clinician is alerted as to the occurrence of a fluctuation in compliance and/or offered additional information regarding one or more potential causes for the fluctuation in compliance and/or offered one or more recommendations for addressing the fluctuation in compliance, as described herein.

### Ventilator GUI Display of Smart Prompt

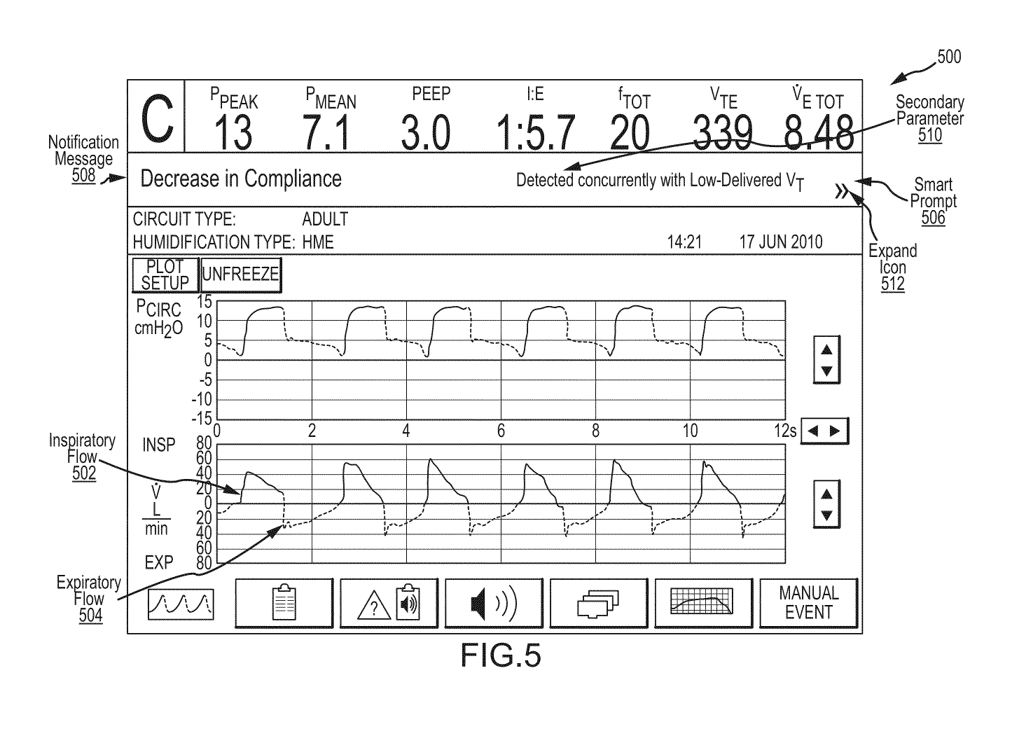

FIG. 5 is an illustration of an embodiment of a graphical user interface displaying a smart prompt element in a window having a notification regarding a fluctuation in compliance and regarding a potential cause for the fluctuation in compliance.

Graphical user interface 500 may display various monitored and/or derived data to the clinician during ventilation of a patient. In addition, graphical user interface 500 may display various messages to the clinician (e.g., alarm messages, etc.). Specifically, graphical user interface 500 may display a smart prompt as described herein.

According to embodiments, the ventilator may monitor and evaluate various ventilatory parameters to detect a fluctuation in compliance. As illustrated, a flow waveform may be generated and displayed by the ventilator on graphical user interface 500. As further illustrated, the flow waveform may be displayed such that inspiratory flow 502 is represented in a different color (e.g., green) than expiratory flow 504 (e.g., yellow). According to embodiments, a fluctuation in compliance may be determined at the end of inspiration, and may be calculated via any means described herein or otherwise known or developed in the future. One or more additional ventilatory parameters may also be calculated or derived. For instance, a delivered V_{T} may be determined via any suitable means, either currently known or developed in the future. According to embodiments, delivered V_{T} may be compared to a threshold V_{T} and, when delivered V_{T} is less than the threshold V_{T}, the ventilator may determine one or more possible causes for the decrease in compliance. According to some embodiments, when a decrease in compliance is detected in conjunction with a delivered V_{T t} that is less than the threshold V_{T} for a period of time or over a number of breaths, the ventilator may provide a notification and/or a recommendation.

According to embodiments, smart prompt 506 may be displayed in any suitable location such that a clinician may be alerted regarding a detected patient condition, but while allowing other ventilatory displays and data to be visualized substantially simultaneously. As illustrated, smart prompt 506 is presented as a bar or banner across an upper region of the graphical user interface 500. However, as previously noted, smart prompt 506 may be displayed as a tab, icon, button, banner, bar, or any other suitable shape or form. Further, smart prompt 506 may be displayed in any suitable location within the graphical user interface 500. For example, smart prompt 506 may be located along any border region of the graphical user interface 500 (e.g., top, bottom, or side borders) (not shown), across an upper region (shown), or in any other suitable location. Further, as described herein, smart prompt 506 may be partially transparent (not shown) such that ventilatory displays and data may be at least partially visible behind smart prompt 506.

Specifically, smart prompt 506 may alert the clinician that a fluctuation in compliance has been detected in conjunction with one or more other ventilatory parameters, for example by notification message 508. For instance, as described herein, notification message 508 may alert the clinician that a decrease in compliance was detected via any suitable means, e.g., “Decrease in Compliance” (shown) or “Increase in Compliance Detected” (not shown). Smart prompt 506 may further include information regarding one or more potential causes for a decrease in compliance occurring with a change in one or more other ventilatory (e.g., increased PIP, or low-delivered V_{T}), e.g., secondary parameter 510. For example, if a decrease in compliance was detected concurrent with low-delivered V_{T}, this information may be provided to the clinician (e.g., “Detected concurrently with low-delivered V_{T},” shown). Alternatively, additional information regarding a potential cause may be provided to the clinician (e.g., “Detected 25% decrease in compliance concurrently with low-delivered V_{T},” not shown; or “Detected 25% decrease in compliance concurrently with low-delivered V_{T}, from start of ventilation,” not shown). According to the illustrated embodiment, secondary parameter 510 is provided along with the notification message 508 in a banner. According to embodiments the illustrated embodiment may correspond to a primary prompt. According to alternative embodiments, in addition to the notification message 508 and the secondary parameter 510, one or more recommendations may be provided in an initial smart prompt banner (not shown). According to other embodiments, rather than providing information regarding one or more potential causes for the fluctuation in compliance in the initial smart prompt (e.g., primary prompt), this information may be provided within an expanded portion (e.g., secondary prompt, not shown) of smart prompt 506.

According to embodiments, smart prompt 506 may be expanded to provide additional information and/or recommendations to the clinician regarding a detected patient condition. For example, an expand icon 512 may be provided within a suitable area of the smart prompt 506. According to embodiments, upon selection of the expand icon 512 via any suitable means, the clinician may optionally expand the smart prompt 506 to acquire additional information and/or recommendations for addressing the detected patient condition. According to further embodiments, smart prompt 506 may include links (not shown) to additional settings and/or display screens of the graphical user interface 500 such that the clinician may easily and quickly address and/or verify the detected condition.

As may be appreciated, the disclosed data, graphics, and smart prompt illustrated in graphical user interface 500 may be arranged in any suitable order or configuration such that information and alerts may be communicated to the clinician in an efficient and orderly manner. The disclosed data, graphics, and smart prompt are not to be understood as an exclusive array, as any number of similar suitable elements may be displayed for the clinician within the spirit of the present disclosure. Further, the disclosed data, graphics, and smart prompt are not to be understood as a necessary array, as any number of the disclosed elements may be appropriately replaced by other suitable elements without departing from the spirit of the present disclosure. The illustrated embodiment of the graphical user interface 500 is provided as an example only, including potentially useful information and alerts that may be provided to the clinician to facilitate communication of detected a fluctuation in compliance in an orderly and informative way, as described herein.

### Ventilator GUI Display of Expanded Smart Prompt

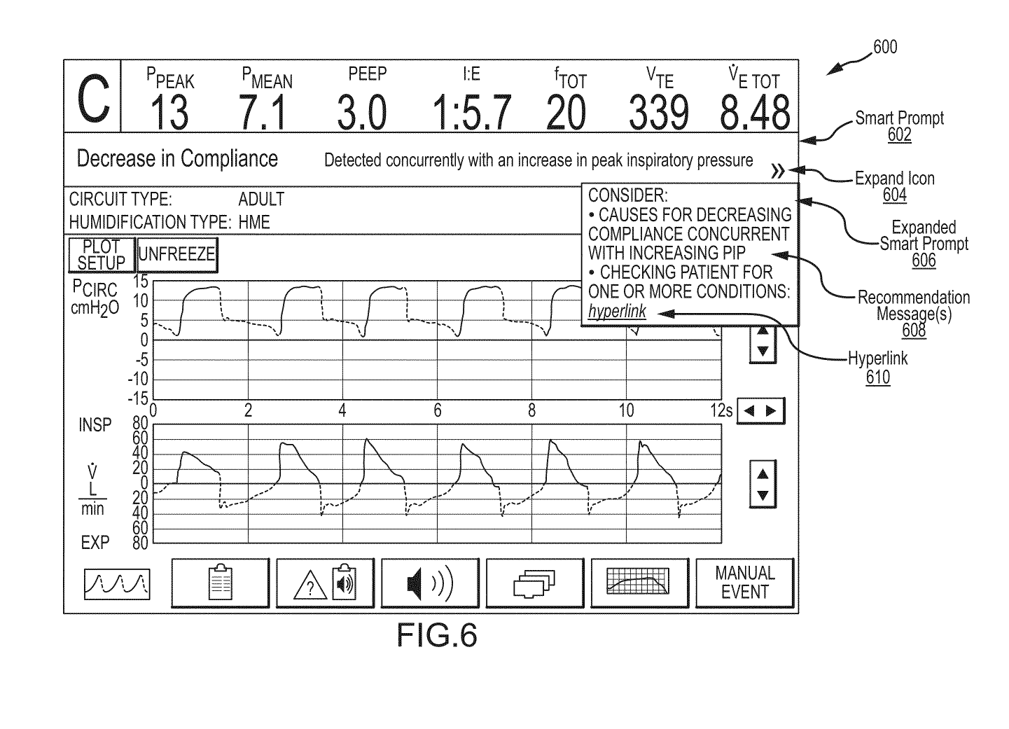

FIG. 6 is an illustration of an embodiment of a graphical user interface displaying an expanded smart prompt element in a window having a notification message regarding a fluctuation in compliance and a recommendation message regarding addressing the fluctuation in compliance

Graphical user interface 600 may display various monitored and/or derived data to the clinician during ventilation of a patient. In addition, graphical user interface 600 may display an expanded smart prompt including one or more recommendation messages, as described herein.

According to embodiments, as described above, an expand icon 604 may be provided within a suitable area of a smart prompt 602. Upon selection of the expand icon 604, the clinician may optionally expand smart prompt 602 to acquire additional information and/or recommendations for addressing the detected patient condition. For example, expanded smart prompt 606 may be provided upon selection of expand icon 604. As described above for smart prompt 506, expanded smart prompt 606 may be displayed as a tab, icon, button, banner, bar, or any other suitable shape or form. Further, expanded smart prompt 606 may be displayed in any suitable location within the graphical user interface 600. For example, expanded smart prompt 606 may be displayed below (shown) smart prompt 602, to a side (not shown) of smart prompt 602, or otherwise logically associated with smart prompt 602. According to other embodiments, an initial smart prompt may be hidden (not shown) upon displaying expanded smart prompt 606. Expanded smart prompt 606 may also be partially transparent (not shown) such that ventilatory displays and data may be at least partially visible behind expanded smart prompt 606. According to some embodiments, expanded smart prompt 606 corresponds to a secondary prompt.

According to embodiments, expanded smart prompt 606 may comprise additional information (not shown) and/or one or more recommendation messages 608 for addressing the fluctuation in compliance. In some instances, the one or more recommendation messages 608 may be based on a type of ventilation (e.g., VC, PC, PS, VC+, VS, or PA ventilation) being delivered to the patient. Furthermore, the one or more recommendation messages 608 may be based on one or more potential causes for the fluctuation in compliance.

For example, during PC or PS ventilation (shown), when low-delivered V_{T} was detected concurrent with a decrease in compliance (not shown), the ventilator may provide the recommendation: “Consider checking patient for barotrauma.” Likewise, in VC or VC+ ventilation, when a decrease in compliance was detected concurrent with an increase in PIP (shown), the ventilator may provide the same recommendation.

According to embodiments, expanded smart prompt 606 may also include one or more hyperlinks 610, which may provide immediate access to the display and/or settings screens associated with a detected fluctuation in compliance. For example, associated parameter settings screens may be accessed from expanded smart prompt 606 via hyperlink 610 such that the clinician may address a detected fluctuation in compliance by adjusting one or more parameter settings, checking the patient, or performing other operations as necessary. For example, hyperlink 610 may be linked to a setup screen such that the clinician or the ventilatory system may modify one or more ventilatory settings. Alternatively, associated parameter display screens may be accessed such that the clinician may view clinical data associated with a fluctuation in compliance in the form of charts, graphs, or otherwise. That is, according to embodiments, the clinician may access the ventilatory data that implicated one or more potential causes for the fluctuation in compliance (e.g., for verification purposes or otherwise). For example, hyperlink 610 may be linked to one or more parameter display screens for evaluating causes for low-delivered V_{T}, for evaluating ventilatory data implicating an increase in PIP, for evaluating ventilatory data implicating an increase in MAP, etc.

As may be appreciated, the disclosed smart prompt and recommendation messages illustrated in graphical user interface 600 may be arranged in any suitable order or configuration such that information and alerts may be communicated to the clinician in an efficient and orderly manner. Indeed, the illustrated embodiment of the graphical user interface 600 is provided as an example only, including potentially useful information and recommendations that may be provided to the clinician to facilitate communication of suggestions for addressing detected fluctuations in compliance in an orderly and informative way, as described herein.

The below table (Table 1) illustrates exemplary conditions and corresponding recommendations that may be displayed in response to one or more detected conditions:

TABLE 1
Recommendation messages based on detected fluctuations in resistance and
compliance according to breath type and secondary conditions.

| | | | Detected | | Primary |
|---|---|---|---|---|---|
| | Detected | | Secondary | Notification | Recommendation |
| Breath Type | Parameter | And | Parameter | Message | Message |
| PS or PC | Airway | Humid Type = | Decreasing Vₜₑ, | Decrease in | Increased AW |
| | Resistance | HME | increase in | Dynamic | (airway) Resistance |
| | increased by | | R_{Dyn}, increased | Compliance | detected. Check |
| | 25% | | EEF, decreased | Detected | airway, HME for |
| | | | PEF | | obstruction, need for |
| | | | | | suctioning, need for |
| | | | | | bronchodilator |
| PS or PC | Airway | Humid Type = | Decreasing Vₜₑ, | Decrease in | Increased AW |
| | Resistance | heated | increase in | Dynamic | (airway) Resistance |
| | increased by | expiratory tube | R_{Dyn}, increased | Compliance | detected. Check |
| | 25% | or non-heated | EEF, decreased | Detected | airway, need for |
| | | expiratory tube | PEF, | | suctioning, need for |
| | | | | | bronchodilator |
| VS, VC, or | Airway | Humid Type = | Increase in PIP, | Decrease in | Increased AW |
| VC+ | Resistance | HME | increase in | Dynamic | (airway) Resistance |
| | increased by | | R_{Dyn}, increased | Compliance | detected. Check |
| | 25% | | EEF, decreased | Detected | airway, HME for |
| | | | PEF | | obstruction, need for |
| | | | | | suctioning, need for |
| | | | | | bronchodilator |
| VS, VC, or | Airway | Humid Type = | Increase in PIP, | Decrease in | Increased AW |
| VC+ | Resistance | heated | increase in | Dynamic | (airway) Resistance |
| | increased by | expiratory tube | RDyn, | Compliance | detected. Check |
| | 25% | or non-heated | increased EEF, | Detected | airway, need for |
| | | expiratory tube | decreased PEF | | suctioning, need for |
| | | | | | bronchodilator |
| PS or PC | Airway | Carinal | Decreasing | Decrease in | Increased AW |
| | resistance | pressure lower | Vte, increase in | Dynamic | (airway) Resistance |
| | increased by | than circuit | RDyn, | Compliance | detected. Check |
| | 25% | pressure more | increased EEF, | Detected | airway, HME for |
| | | than expected | decreased PEF, | | obstruction, need for |
| | | | carinal pressure | | suctioning |
| | | | lower than | | |
| | | | circuit pressure | | |
| | | | more than | | |
| | | | expected | | |
| VC+, VS or | Airway | Carinal | Increasing PIP, | Decrease in | Increased AW |
| VC | resistance | pressure lower | increase in | Dynamic | (airway) Resistance |
| | increased by | than circuit | RDyn, | Compliance | detected. Check |
| | 25% | pressure more | increased EEF, | Detected | airway, HME for |
| | | than expected | decreased PEF, | | obstruction, need for |
| | | | carinal pressure | | suctioning |
| | | | lower than | | |
| | | | circuit pressure | | |
| | | | more than | | |
| | | | expected | | |
| PS or PC | Expiratory | N/A | Decreasing V | Decrease in | Increase expiratory |
| | circuit R | | Vte, increase in | Dynamic | Circuit Resistance |
| | increased by | | Circuit RDyn, | Compliance | detected: Check |
| | 25% | | increased EEF, | Detected | expiratory filter, |
| | | | decreased PEF, | | partial tubing |
| | | | increased MAP | | obstruction, and |
| | | | | | condensate collection |
| VC+, VS or | Expiratory | N/A | Increasing PIP, | Decrease in | Increase expiratory |
| VC | circuit R | | increase in | Dynamic | Circuit Resistance |
| | increased by | | Circuit RDyn, | Compliance | detected: Check |
| | 25% | | increased EEF, | Detected | expiratory filter, |
| | | | decreased PEF, | | partial tubing |
| | | | increased MAP | | obstruction, and |
| | | | | | condensate collection |
| PS or PC | Static | N/A | Decreasing V | Decrease in | Decreased compliance |
| | compliance | | Vte, decrease in | Static | detected: check |
| | decreased by | | C_{Dyn}, | Compliance | patient for |
| | 25% | | | detected | barotrauma, fluid in |
| | | | | | lungs, worsening |
| | | | | | pneumonia, ETT in |
| | | | | | mainstem, pleural |
| | | | | | effusion, potential for |
| | | | | | ALI or ARDS. |
| VC+, VS or | Static | N/A | Increasing PIP, | Decrease in | Decreased compliance |
| VC | compliance | | increased MAP, | Static | detected: check |
| | decreased by | | decrease C_{Dyn}, | Compliance | patient for |
| | 25% | | | detected | barotrauma, fluid in |
| | | | | | lungs, worsening |
| | | | | | pneumonia, ETT in |
| | | | | | mainstem, pleural |
| | | | | | effusion, potential for |
| | | | | | ALI or ARDS. |
| VC+, VS or | Airway | Carinal | increasing PIP, | Decrease in | Increased AW |
| VC | Resistance | pressure is | increased MAP, | Dynamic | (airway) Resistance |
| | increased by | lower than | increase in | Compliance | detected. Check |
| | 25% | circuit pressure | R_{Dyn}, increased | Detected | airway, HME for |
| | | than expected | EEF, decreased | | obstruction, need for |
| | | | PEF | | suctioning |
| PS or PC | Airway | Carinal | Decreasing V | Decrease in | Increased AW |
| | Resistance | pressure is | Vₜₑ, increased | Dynamic | (airway) Resistance |
| | increased by | lower than | MAP, increase | Compliance | detected. Check |
| | 25% | circuit pressure | in R_{Dyn}, | Detected | airway, HME for |
| | | than expected | increased EEF, | | obstruction, need for |
| | | | decreased PEF | | suctioning |
| VC, VS or | Static | C20/C | Increasing PIP, | Decrease in | Consider lowering |
| VC+ | compliance | decreased by | decrease in | Static | tidal volume setting |
| | decreased by | 25% and high | C_{Dyn}, increased | Compliance | |
| | 25% | tidal volume/kg | MAP | detected | |
| | | of PBW | | | |
| PS or PC | Static | C20/C | Decreasing V | Decrease in | Consider lowering PI |
| | compliance | decreased by | Vₜₑ, decrease in | Static | setting |
| | decreased by | 25% and high | C_{Dyn}, increased | Compliance | |
| | 25% | tidal volume/kg | MAP | detected | |
| | | of PBW | | | |
| VC, VC+, PS, | Static | C20/C | Decreasing Vte, | Decrease in | Consider addressing |
| or PC | compliance | decreased by | decrease in | Static | causes of Auto-PEEP |
| | decreased by | 25% and Auto- | C_{Dyn}, | Compliance | to improve |
| | 25% | PEEP detected | | detected | compliance |
| VC and flow | Static | Lower | Increasing PIP, | Decrease in | Consider increasing |
| pattern is | compliance | inflection point | decrease in | Static | set PEEP to address |
| square | decreased by | detected | C_{Dyn}, increased | Compliance | lower inflection point |
| | 25% | | MAP, lower | detected | |
| | | | inflection on | | |
| | | | pressure- | | |
| | | | volume curve | | |
| VC, VC+, PS, | Static | Asynchronous | Detection of | Decrease in | Consider |
| or PC | compliance | chest | asynchronous | Static | pneumothorax |
| | decreased by | movement | movement of | Compliance | |
| | 25% | detected and | left and right | detected | |
| | | tube type is | chest from chest | | |
| | | Tracheostomy | sensors | | |
| VC, VC+, PS, | Static | Asynchronous | Detection of | Decrease in | Consider mainstem |
| or PC | compliance | chest | asynchronous | Static | intubation or |
| | decreased by | movement | movement of | Compliance | pneumothorax |
| | 25% | detected and | left and right | detected | |
| | | tube type is | chest from chest | | |
| | | Endotracheal | sensors | | |

Unless otherwise indicated, all numbers expressing measurements, dimensions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. Further, unless otherwise stated, the term “about” shall expressly include “exactly,” consistent with the discussions regarding ranges and numerical data. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 4 percent to about 7 percent” should be interpreted to include not only the explicitly recited values of about 4 percent to about 7 percent, but also include individual values and sub-ranges within the indicated range. Thus, included in this numerical range are individual values such as 4.5, 5.25 and 6 and sub-ranges such as from 4-5, from 5-7, and from 5.5-6.5, etc. This same principle applies to ranges reciting only one numerical value. Furthermore, such an interpretation should apply regardless of the breadth of the range or the characteristics being described.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the appended claims.

What is claimed is:

1. A ventilator-implemented method for detecting a fluctuation in compliance, the method comprising:
receiving one or more ventilatory settings, wherein the one or more ventilatory settings include a baseline compliance;
collecting ventilatory data;
processing the collected ventilatory data, wherein processing the collected ventilatory data includes trending compliance during ventilation of a patient;
analyzing the trended compliance comprising comparing the trended compliance to the baseline compliance;
detecting a decrease in compliance upon determining that the trended compliance is less than the baseline compliance; and
displaying a notification message when the decrease in compliance is detected.

2. The method of claim 1, further comprising:
retrieving patient data, wherein the patient data comprises at least one of: a patient diagnosis, a patient predicted body weight (PBW), and a patent gender.

3. The method of claim 1, further comprising:
retrieving at least one secondary ventilatory parameter.

4. The method of claim 3, further comprising:
identifying one or more potential causes for the decrease in compliance based at least in part on the occurrence of both the decrease in compliance and the retrieved secondary ventilatory parameter.

5. The method of claim 4, wherein identifying the one or more potential causes for the decrease in compliance further comprises at least one of:
detecting low-delivered tidal volume (V_{T}) concurrently with the decrease in compliance;
detecting an increase in peak inspiratory pressure concurrently with the decrease in compliance; and
detecting an increase in mean airway pressure concurrently with the decrease in compliance.

6. The method of claim 4, wherein displaying the notification message includes displaying the one or more potential causes for the decrease in compliance.

7. The method of claim 6, further comprising:
determining one or more recommendations for addressing the decrease in compliance based on the determined one or more potential causes and at least some patient data; and
displaying the one or more recommendations.

8. The method of claim 7, wherein displaying the one or more recommendations further comprises:
displaying an icon for accessing the one or more recommendations, wherein upon activating the icon the one or more recommendations are displayed.

9. The method of claim 5, further comprising:
displaying an option to disable activation of the icon.

10. A ventilatory system for issuing a prompt when a decrease in compliance is detected, comprising:
at least one processor; and
at least one memory, communicatively coupled to the at least one processor and containing instructions that, when executed by the at least one processor, perform a method comprising:
detecting a decrease in compliance;
identifying one or more secondary ventilatory parameters occurring in conjunction with the decrease in compliance;
determining one or more recommendations for addressing the decrease in compliance, at least partially based on the identified secondary ventilatory parameter; and
displaying a prompt comprising one or more of:
an alert regarding the decrease in compliance;
a notification message displaying the one or more secondary ventilatory parameters occurring in conjunction with the decrease in compliance; and
a recommendation message displaying the one or more recommendations for addressing the decrease in compliance.

11. The ventilatory system of claim 10 wherein detecting the decrease in compliance further comprises:
retrieving compliance data;
trending the compliance data over a time period;
comparing the trended compliance data to a compliance threshold; and
determining that the compliance data breaches the compliance threshold.

12. The ventilatory system of claim 10, wherein the prompt comprises:
a primary prompt that includes the alert regarding the a decrease in compliance and the notification message displaying the one or more detected secondary ventilatory parameters; and
a secondary prompt that includes the recommendation message displaying the one or more recommendations for addressing the decrease in compliance.

13. The ventilatory system of claim 10, wherein the notification message displays at least one of: a decrease in compliance detected concurrently with an increase in peak inspiratory pressure and a decrease in compliance detected concurrently with low-delivered tidal volume (V_{T}).

14. The ventilatory system of claim 10, wherein the recommendation message comprises one or more of:
consider checking patient for barotrauma; and
consider checking patient for fluid in lungs.

15. The ventilatory system of claim 10, wherein the recommendation message comprises one or more of:
consider checking for worsening pneumonia;
consider checking for endotracheal tube displacement; and
consider checking for pleural effusion.

16. A ventilator-implemented method for detecting a fluctuation in compliance, the method comprising:
receiving one or more ventilatory settings, wherein the one or more ventilatory settings include a baseline compliance;
collecting ventilatory data;
processing the collected ventilatory data, wherein processing the collected ventilatory data includes trending compliance during ventilation of a patient;
analyzing the trended compliance comprising comparing the trended compliance to the baseline compliance;
detecting an increase in compliance upon determining that the trended compliance is more than the baseline compliance; and
displaying a notification message when the increase in compliance is detected.

17. The method of claim 16, further comprising:
retrieving at least one secondary ventilatory parameter; and
identifying one or more potential causes for the increase in compliance based at least in part on the occurrence of both the increase in compliance and the retrieved secondary ventilatory parameter.

18. The method of claim 17, wherein identifying the one or more potential causes for the increase in compliance further comprises at least one of:
detecting high-delivered tidal volume (V_{T}) concurrently with the increase in compliance;
detecting a decrease in peak inspiratory pressure concurrently with the increase in compliance; and
detecting a decrease in mean airway pressure concurrently with the increase in compliance.

19. The method of claim 18, further comprising:
determining one or more recommendations for addressing the increase in compliance based on the determined one or more potential causes and at least some patient data; and
displaying the one or more recommendations, further including displaying an icon for accessing the one or more recommendations, wherein upon activating the icon the one or more recommendations are displayed.

20. The ventilatory system of claim 19, wherein the notification message displays: an increase in patient inspiratory effort detected concurrently with the high-delivered V_{T}, and wherein the recommendation message comprises one or more of:
consider causes for the increase in patient inspiratory effort; and
consider increasing set V_{T}.
